(19) United States
(12) Patent Application Publication
(10) Pub. No.: US 2003/0125262 A1
(43) Pub. Date: Jul. 3, 2003

(54) METHODS AND REAGENTS FOR REGULATION OF CELLULAR RESPONSES IN BIOLOGICAL SYSTEMS

(75) Inventors: Dr. Laura L. Kiessling, Madison, WI (US); Dr. Laura E. Strong, Madison, WI (US); Dr. Jason E. Gestwicki, Madison, WI (US)

Correspondence Address: GREENLEE, WINNER & SULLIVAN, Sally A. Sullivan, 5370 Manhattan Circle, Suite 201, Boulder, CO, 80303, US, winner@greenwin.com, 303-499-8080, 303-499-8089

(73) Assignee: WISCONSIN ALUMNI RESEARCH FOUNDATION, MADISON, 53707-7365

(21) Appl. No.: 09/815,296

(22) Filed: Mar. 21, 2001

Related U.S. Application Data
(60) Provisional application No. 60/191,014, filed on Mar. 21, 2000.

(51) Int. Cl. A61K 31/70; A61K 38/00

(52) U.S. Cl. 514/23; 514/2

(57) ABSTRACT

### Abstract of Disclosure

This invention provides multivalent ligands which carry or display at least one recognition element (RE), and preferably a plurality of recognition elements, for binding directly or indirectly to cells or other biological particles or more generally by binding to any biological molecule. The multivalent ligands provided can most generally function for binding or targeting to any biological particle or molecule and particularly to targeting of cells or cell types or viruses, for cell aggregation and generally for macromolecular assembly of biological macromolecules. The multivalent ligands of this invention are generally applicable for creating scaffolds (assemblies) of chemical or biological species, including without limitation, antigens, epitopes, ligand binding groups, ligands for cell receptors (cell surface receptors, transmembrane receptors and cytoplasmic receptors), and various macromolecules (nucleic acids, carbohydrates, saccharides, proteins, peptides, etc.). In these scaffolds, the number, spacing, relative positioning and relative orientation of recognition elements can be controlled. Multivalent ligands of this invention can carry or display at least one signal recognition element (SRE), and preferably a plurality of signal recognition elements, and modulate biological responses in biological systems. The invention also relates to methods for aggregating biological particles and macromolecules and for modulating biological response employing the multivalent ligands provided.

### Cross Reference to Related Applications

This application takes priority under 35 U.S.C. 119(d) from U.S. provisional application serial number 60/191,014 filed March 21, 2000 which is incorporated by reference herein to the extent that it is not inconsistent with the disclosure herein.

### Statement Regarding U.S. Government Funding

This invention was made at least in part with funding from the United States government through National Institute of Health grant GM55984. The United States government has certain rights in this invention.

### Background of Invention

A variety of biological processes are mediated by the binding of one chemical or biological species, macromolecule or particle (e.g., a cell, virus or virion) to another chemical or biological species, macromolecule or particle. In many cases there is evidence that the valency of the binding may be an important aspect of the mechanism of the mediation of the biological process. The present invention relates to compounds and methods for selectively varying the valency of such interactions employing multivalent ligands to which a plurality of chemical or biological species involved in binding to other chemical or biological species are (generally designated recognition elements, RE, herein) are attached in a controlled fashion, with control over the number of RE, the spacing of RE and the relative orientation of RE. Certain recognition elements are involved directly or indirectly in biological signaling processes. Other recognition elements are involved simply in facilitating binding that is associated with the biological process. This invention then is generally related to the control of biological processes by controlling the structure of such multivalent ligands. The multivalent ligands of this invention have applications particularly in cell signaling processes and more generally in macromolecular assembly of recognition elements that are involved in biological processes.

Cells need to continuously sense and respond to changes in their environment. For this purpose, cells use a multitude of cell surface, transmembrane and cytoplasmic receptors. These receptors typically recognize proteins, peptides, saccharides, nucleic acids, or other small molecules but, in some cases, receptors may also recognize changes in redox potential, temperature, and osmolarity. The binding of a ligand to these receptors results in changes in the activity of the cell such as migration, activation, metabolism, protein production, differentiation, proliferation, and cell death. This is a central paradigm of cell biology and these cellular responses allow the cell (or the multicellular organism) to properly respond to environmental changes.

The mechanisms by which ligands promote cellular processes are of great interest to elucidate their roles in the regulation of cellular responses. One common way in which these systems are regulated is by the spatial organization of the receptors. Ligand binding can change the relative orientations and/or conformations of the cell surface receptors, activating a response. Biological responses ranging from immune recognition, cell adhesion and migration, and proliferation, among others, rely on the reorientation or change in distribution (e.g., localization) of cell surface receptors that occurs upon ligand binding. Ligand reorientation can be the event that transmits signals and facilitates the cellular response.

A common example of this is found in the growth factor receptors, which govern cell proliferation. Certain divalent growth factors, such as erythropoeitin (EPO), bind to two cell surface growth factor receptors (EPOR) simultaneously and bring those receptors into proximity. This ligand reorganization triggers a signal transduction cascade that involves cross-phosphorylation of the receptors in the dimer. In the EPO example a ligand, which is only capable of binding to one receptor, is incapable of eliciting the response.

A particularly interesting feature of some ligands is valency, which herein refers generally to an interplay between the net number of recognition sites in a ligand for binding to receptors (e.g., epitopes) and the density and spacing of those sites in the ligand. Ligands often possess multiple receptor binding sites. This allows multivalent interactions between the ligand and multiple receptors which may determine the kind and intensity of biological response to that ligand. Often in these systems, monovalent ligands lack any biological activity. Researchers have explored ligands which vary in valency, at least in the sense of increasing the number of recognition sites. Typically the ligands examined have been either small, low valency compounds, such as antibodies or dimerizing agents, or large heterogeneous compounds, such as protein conjugates, polymers, or functionalized surfaces. Work with defined low valency compounds has led to the realization of the extent of regulation by changes in receptor orientation and work with large undefined multivalent ligands has indicated that increasing the net number of recognition sites (e.g., epitopes) can often result in increased effects in many systems.

Cells require fine control over their cellular processes in order to avoid over- or under-stimulation. In the immune response, for example, immune cell function must be closely regulated to avoid unfavorable autoreactivity or clonal anergy. Cells utilize features of the interaction of receptors with ligands to regulate their responses. For example, increased synthetic ligand density has been shown to more effectively activate the response of certain cells to the ligand. Nature may utilize ligand valency to control biological responses in a defined manner. Thus, selective control of biological responses may be achieved though control of ligand valency. Previously described multivalent ligands have, however, not allowed exploration of this fine-tuned control in biological systems.

This invention provides for the generation of synthetic ligands with distinct valencies and controlled features which can be used to systematically alter and/or control biological responses initiated or triggered by binding to cell surface receptors. In particular, the synthetic ligands of this invention allow for access to the finer control exhibited by natural ligands. Access to these features in a synthetic ligand not only expands our understanding of the natural function of these systems, but also leads to selectively designed effector molecules (multivalent ligands) for use in therapeutic and non-therapeutic applications that take advantage of the ability to regulate a wide variety of biological responses.

### Summary of Invention

This invention provides multivalent ligands which carry or display at least one recognition element (RE), and preferably a plurality of recognition elements, for binding directly or indirectly to cells or other biological particles or more generally for binding to any biological molecule. The multivalent ligands provided can most generally function for binding or targeting to any biological particle or molecule and particularly for targeting of cells or cell types or viruses, for cell aggregation and generally for macromolecular assembly of biological macromolecules. The multivalent ligands of this invention are generally applicable for creating scaffolds (assemblies) of chemical or biological species, including without limitation, antigens, epitopes, ligand binding groups, ligands for cell receptors (cell surface receptors, transmembrane receptors and cytoplasmic receptors), and various macromolecules (nucleic acids, carbohydrates, saccharides, proteins, peptides, etc.). In these scaffolds, the number, spacing, relative positioning and relative orientation of recognition elements can be controlled.

In a more specific embodiment, multivalent ligands are provided which carry or display at least one signal recognition element (SRE), and preferably a plurality of signal recognition elements, and modulate biological responses in biological systems. Signal recognition elements provide for binding to a cell surface receptor and alone or in combination with other SRE affect a biological response in a biological system. SRE include chemical or biochemical species recognized as signals by a cell, i.e., through binding one or more cell receptors, particularly one or more cell surface receptors. These multivalent ligands can act generally as effectors of biological responses in biological systems. The multivalent ligands provided can function to activate, initiate or trigger a biological response, to inhibit a response, to enhance or attenuate a response, or to change the nature of a response. A multivalent ligand of this invention can also affect a response mediated through a cell surface receptor to which it does not itself bind.

The invention provides methods for labeling or targeting of cells with functional elements (FE). The invention also provides methods for inducing or enhancing cell aggregation or alternatively for inhibiting or preventing cell aggregation. The invention further provides methods for inducing, modulating and/or regulating biological responses in biological systems. Each of these methods employ multivalent ligands. Preferred multivalent ligands of this invention have defined or controlled valency, in which structural features of the ligand are selected or controlled, including the number, density, spacing and orientation of recognition elements (RE and SRE) for binding to receptors, to simply bind to a cell or to obtain a desired type of biological response or level of response.

Scaffolded multivalent ligands of this invention which comprise a plurality of RE, SRE or both optionally in combination with FE can be employed in a variety of diagnostic and clinical applications, in particular in blood typing and in pathogen detection. The multivalent ligands herein can be employed in the detection of various biological molecules and particles (cells and viruses) and in a variety of assay methods(histology, Western blots, PCR assays, ELISA assays, agglutination assays, among others). In general, increases in valency in such ligands will be associated with an increase in assay or diagnostic sensitivity.

Multivalent ligands comprise one or more structural or functional groups which act as recognition elements (RE) for binding to cell surface receptors, optionally in combination with one or more signal recognition elements (SRE), or one or more functional elements (FE), or both. SREs are a subset of REs that, alone or in combination with other SREs (REs or FEs) in a multivalent ligand, can induce intracellular and/or intercellular biological responses. Multivalent ligands of this invention carrying one or more SRE (optionally in combination with one or more RE, one or more different SRE or one or more FE) can initiate a biological response in a cell. Alternatively, these multivalent ligands can modulate the response of a cell in the presence of one or more natural chemical or biochemical signals, for example, by enhancing, decreasing or inhibiting the response. In specific embodiments, multivalent ligands of this invention are designed to change the level or type of response that is induced in a cell by a selected chemical or biochemical signal.

Multivalent ligands of this invention most generally comprise a molecular scaffold to which a plurality of REs, SREs or both (optionally in combination with FEs) are bonded either by covalent or non-covalent interactions. The number, density and spacing of the RE, SRE and FE on the scaffold can be controlled, typically by selective synthesis of desired ligands. The molecular scaffold can be linear, branched or cyclic providing different geometries of presentation of RE and/or SREs to cells. In preferred embodiments, molecular scaffolds are polymers comprising a plurality of monomers. Molecular scaffold of the multivalent ligands of this invention include polymers in which all of the monomers are the same or copolymers containing a mixture of different monomers. Molecular scaffolds can also include block copolymers in which different regions (or portions) of the scaffold are composed of different monomers. Molecular scaffolds prepared by ROMP methods, as illustrated in several formulas herein, are preferred.

Molecular scaffolds can be hydrophobic or can be made to be more hydrophilic by substitution (particularly of the polymer backbone) with polar substituents, such as -OH. The scaffold can be substituted, in general, with any groups that do not interfere with RE or SRE activity, e.g. binding to a receptor. Substitution of the scaffold can be controlled to adjust the physical properties, e.g., solubility, of the multivalent ligand. REs, SREs and FEs may be directly attached to a scaffold or attached to the scaffold via linker groups. The linker group provides functional groups for bonding to the scaffold and for bonding to REs, SREs and/or FEs and can also affect solubility of the multivalent ligand. The linker can also provide a defined spacer to minimize undesired interactions among REs, SREs or FEs or between the attached elements and the scaffold or to provide structural flexibility with respect to orientation of attached elements.

In specific embodiments, the molecular scaffold comprises a plurality of repeated units (monomers) to each of which an RE or SRE is bonded. In general, the molecular scaffold functions to hold the signals in proximity to each other and does not interact directly in the modulation of the biological response. However, physical (e.g., solubility) or chemical (e.g., stability) properties of the multivalent ligands can be varied by selection of the structure of the scaffold or by introducing substituents (e.g., polar, non-polar) along the scaffold.

In one embodiment, the multivalent ligands have only one type of RE or SRE in the ligand. These multivalent ligands include dimers, trimers, tetramers and polymers (including relatively short oligomers having 5 or more monomers) or longer polymers having 25, 50, 100 or more monomers. Preferred multivalent ligands carrying one type of RE or SRE carry about 10 or more of such REs or SREs. In this embodiment, the repeating units (or monomers) of the multivalent ligand are preferably the same.

In another embodiment, the invention provides multivalent ligands that carry more than one type of RE, more than one type of SRE or a combination of RE and SRE. These multivalent ligands also include dimers (carrying one of each RE or SRE or an RE and an SRE), trimers, tetramers and block polymers (including relatively short oligomers having 5 monomers or more)or longer polymers having 25, 50, 100 or more monomers. These multivalent ligands may also have spacer regions (with monomers that do not carry any RE or SRE group) along the scaffold to separate regions carrying a first RE or SRE from regions carrying a second RE or SRE. Monomers in spacer regions may carry a functional element (FE), may be unsubstituted or may carry a non-reactive, non-functional group. A given multivalent ligand can generally contain any number of different REs, SREs, or both, however those carrying 2 or 3 different RE or SRE are of most interest.

In other embodiments, the invention provides multivalent ligands that carry one or more RE or SRE, which may be the same or different, but also carry functional elements other than RE or SRE. These functional elements (FE) can, for example, exhibit a variety of chemical or biochemical functions (different from those of REs or SREs). They can, for example, provide one or more fluoresecent or radiolables, provide one or more groups with latent reactive groups, or provide one or more enzymatic functions. Substitution of monomers with FEs can also provide for spacing of SREs.

Recognition elements (RE) are any chemical or biological species (e.g., molecules or portions thereof) that alone or in combination with one or more other REs, recognize and bind to a cell surface receptor. RE can, for example, include all or a portion of a ligand active for binding to a cell surface receptor. Signal recognition elements (SRE) are any chemical or biochemical species that, alone or in combination with one or more other SREs, induce a biological response in or from a cell and include biological molecules (proteins, glycoproteins, peptides, amino acids, nucleic acids, saccharides, cytokines, growth factors, hormones, and various derivatives thereof) and which may be portions of larger biological species (protein fragments, epitopes, antigenic determinant, etc.) and various chemical species (haptens, naturally-occurring drugs, synthetic drugs) and species that act as functional mimics of biological molecules (e.g., peptoids, phosphorothioates). SRE are typically RE which bind to a cell surface receptor, but in contrast to RE, SRE affect a biological response in the cell.

Multivalent ligands of this invention can function to reorganize and/or cluster cell receptors. In this regard the RE or SRE on the multivalent ligand will be a ligand of the cell receptor. In certain cases, clustering or reorganization of receptors modulates the cell"s response to a given SRE. Clustering or reorganization of receptors by a multivalent ligand of this invention can also modulate the response of a cell to another signal or another ligand. Through clustering or other structural reorientation or reorganization of cell surface receptors, a multivalent ligand of this invention can enhance or inhibit the cell"s response to another signal or ligand. For example, multivalent ligands of this invention that function as chemoattractants can enhance the response of a cell to another chemoattractant.

A given cell receptor may mediate more than one biological response. The multivalent ligands of this invention that carry ligands which bind to a given cell receptor, but which do not induce a biological response mediated by that receptor, may be employed to inhibit the biological response.

Multivalent ligands that carry more than one type of SRE can be used to simultaneously or sequentially induce more than one biological response in or from a cell. Alternatively, the cellular response to one SRE can be modified by the cellular response to another SRE. Multivalent ligands carrying two or more different SREs can function, for example, to reorganize different receptors on the cell surface, which can result in modulation of cellular response to one or more SREs. Similarly, in multivalent ligands carrying FE, in addition to one or more SRE, the response to an SRE can be modified by the presence of FE.

Multivalent ligands of this invention can be employed in methods to modulate signal transduction processes (i.e., the transmission of information between the outside and the inside of a cell and between cells, in biological systems) in prokaryotic or eukaryotic cells. The methods can be practiced in vivo, in vitro or ex vivo (where cells are removed from a natural environment, including a multicellular organism, and are intended once treated to be returned to that environment). For example, chemotaxis or cell migration responses to SREs can be modulated. Such methods are applicable to prokaryotes (e.g., Gram negative, as well as Gram positive bacteria), eukaryotic microorganisms (including, without limitation, eukaryotic parasites and pathogens of various organisms, including mammals), and eukaryotic cells of larger organisms including those of mammals, and specifically including those of humans (e.g., leukocytes, lymphocytes, endothelial cells, and epithelial cells). Multivalent ligands that modulate responses in bacterial cells or in eukaryotic cells, including eukaryotic pathogens or parasites, can be used to inhibit proliferation, colonization, migration, or biofilm formation by the bacterium, or eukaryotic pathogen or parasite and, as a consequence, can inhibit infection or colonization by such microorganisms.

Multivalent ligands can also be used to promote or inhibit cell differentiation, cell proliferation and/ or cell death (e.g., apoptosis). Multivalent ligands that modulate responses in eukaryotic cells of larger organisms can be used to inhibit undesired cell proliferation, undesired migration and undesired formation of cell to cell junctions or to promote or enhance desired cell proliferation, desired migration and desired formation of cell junctions dependent upon the selection of SRE and other FE in the multivalent ligand.

Pharmaceutical and therapeutic compositions which comprise a pharmaceutically acceptable carrier and an amount of a multivalent ligand effective for modulating cell proliferation, colonization, migration, cell to cell junction formation and/or biofilm formation by eukaryotic or prokaryotic cells are encompassed by this invention. Specific pharmaceutical or therapeutic compositions include those which comprise an amount of a multivalent ligand effective for inhibiting or disrupting undesired cell proliferation, colonization, migration, cell to cell junction formation and/or biofilm formation by eukaryotic or prokaryotic cells. Pharmaceutical compositions that retard or inhibit infections by bacteria or eukaryotic parasites or pathogens are of particular interest. Two or more multivalent ligands of this invention can be combined in such pharmaceutical compositions to provide for combined effect and benefit.

Cell migration, adhesion and the formation of cell to cell junctions are involved in cancer growth and metastasis. Multivalent ligands that modulate such processes can be employed in methods and pharmaceutical compositions for inhibition of cancer growth and metastasis. Again such pharmaceutical compositions include those which comprise an amount of a multivalent ligand that is effective for inhibiting cancer cell growth, adhesion or migration. Two or more multivalent ligands of this invention can be combined in such pharmaceutical compositions to provide for combined effect and benefit.

Multivalent ligands of this invention can modulate immune responses in animals (including mammals and particularly in humans) by valency-dependent interaction with cells that function in the immune system (e.g., leukocytes and lymphocytes). In particular, multivalent ligands of this invention can modulate the response of leukocytes, including neutrophils, to chemoattractants (including derivatized peptides, such as N-formyl peptides, and N-acyl peptides) and can modulate the activation and deactivation of B-cells and/or T-cells. B-cell and/or T-cell activation can be performed in vivo, in vitro and/or ex vivo.The invention also provides libraries of multivalent ligands in which the members of the libraries are varied, for example, in the number and/or relative positioning of RE or SRE, the presence and/or positioning of spacers, in the number of repeating units or monomers (e.g., n or n+m in formulas below) and in the presence or number of FE. Libraries of multivalent ligands which span a range of defined sizes, numbers of repeating units or monomers, numbers of RE or SRE, combinations of RE or SRE, combinations of RE, SRE and FE and spacing of attached elements , (RE, SRE and any FE) are of particular interest. Libraries prepared using ROMP-methods are of particular interest and application. Using various selection and screening methods that are understood in the art, these libraries can be selected or screened for multivalent ligands in the library which exhibit desired modulation in a given biological system. Furthermore, the results obtained from such screens, i.e., the number of RE required for cell aggregation, the number of SRE"s required for induction or inhibition, and other structure/function relationships, can be used in the design and synthesis of additional multivalent ligands.

### Brief Description of Drawings

Figure 1 schematically illustrates several ways in which multivalent ligands of this invention can functions in macromolecular assembly and as effectors of biological responses.

Figure 2A: Results of video microscopy motion analysis experiments. Bacteria (Escherichia coli ) were treated with buffer alone, galactose, or compound 1 or 3 (Scheme 1) at the indicated saccharide concentrations. The results represent the average from at least five independent experiments performed in triplicate. Error bars represent the deviation between per-second averages during the ten second interval.

Figures 2B-E: Selected sample paths for bacteria (Gram Negative, E. coli) treated with buffer alone (B); 1 mM galactose (C); or 1mM compound 1 (D); or 1 mM compound 3 (E). Sample paths are derived from motion of representative bacteria from a treated bacterial population.

Figures 3A and 3B: Results of E. coli capillary accumulation assays. The number of bacteria accumulated is plotted versus the concentration of the attractant (galactose or compounds 1 -4, Scheme 1) calculated on a saccharide residue basis. (A): Results are shown for capillaries filled with buffer alone, compound 1, and compound 2 or (B): buffer alone, compound 3 and compound 4 at the indicated concentrations. The vertical line at 1 mM indicates the concentration of maximum chemotaxis for the monomeric compound 1. The concentrations used in this assay are not directly comparable to those used in the motion analysis experiments (see Figure 2A), because the gradient formed in the capillary assay is not defined. Results are the average of 3 to 6 experiments performed in duplicate and error bars represent a single standard deviation. Partial permeabilization was required to obtain chemotaxis towards 4, and was utilized for all experiments [57].

Figure 4: Results of B. subtilis capillary accumulation assays using ROMP-derived glucose ligands (compound 5 -7, Scheme 1). Buffer alone, glucose, or glucose-bearing compounds 5-7 were used as attractants in the capillary accumulation assay. Results are shown for glucose, compound 5, compound 6, and compound 7. Results are the average of at least four trials performed in duplicate and error bars represent single standard deviations.

Figures 5A and B: Results of video microscopy motion analysis experiments. (A): Bacteria (E. coli) were treated with increasing concentrations of serine (μM) after initial treatment (followed by a 2 min adaptation period) with buffer alone (θ) or 10µM attractant: galactose (Ο), compound 1 (10mer, Δ) or compound 3 (25 mer, ◊); (B) Bar graph of data for angular mean velocity taken from Fig. 6A at serine concentration 1µM. Initial treatment with compound 3 results in a significant enhancement of bacterial response to serine. Angular mean velocities varied approximately 14% between experiments performed on different days.

Figure 6: Multivalent ligands bind specifically to chemoreceptors and induce receptor reorganization. The illustration schematic represents fluorescently labeled 8 (10, 590 nm emission) bound to Trg (11) via GGBP(12). Trg is labeled with anti-Tsr antibody (13, 530 nm emission).

Figures 7A-D: Model of receptor reorganization by synthetic ligands. (A) Chemoreceptors are observed to form dimers (or multimers) (20) in the plasma membrane of E. coli and each dimer appears to interact with a single periplasmic binding protein (21) [59, 60]. Monovalent galactose ligands, such as galactose and compound 1 (22), interact with Trg through GGBP binding, inducing signal transduction from chemoreceptor dimers; (B) Multivalent galactose compounds, such as compound 2, that cannot span the distance needed to reorganize the receptors (23) generate signals from individual dimers, as in (A); (C) Multivalent ligands of sufficient lengths (24), such as compounds 3 and 4, are able to reorganize the chemoreceptors into discrete clusters (25) at the plasma membrane; (D) Extending the valency of a multivalent ligand (26) likely increases the extent of reorganization and, therefore, the bacterial response.

Figure 8: illustrates various designs for molecular scaffolds that can be employed in the multivalent ligands of this invention. These types of scaffolds can be constructed, for example, employing alicyclic or aromatic (including heteroaromatic) ring systems and combinations thereof. Scaffolds provide the geometry of presentation of two or more REs, SREs or both. Linkers may have varying structures and, for example, be rigid, flexible or branched. In each of the illustrated structures any of a rigid, flexible or branched linker can be employed. Each branched linker may be attached to more than one RE, SRE (or FE). In each structure, one or more FE (so long as at least one RE or SRE remains) can replace one or more RE or SRE.

Figures 9A-C: Illustrate models of the ability of multivalent ligands to activate or inhibit cell aggregation in a valency- and concentration-dependent fashion; (A) Monovalent ligands (31) (such as 9) are necessarily inhibitory if they bind to ConA (30); (B) Multivalent ligands 32 (such as 12) at sufficiently low concentrations and optimal stoichiometry with ConA may allow cell aggregation (34), despite their occupation of ConA binding sites; (C) At increased concentrations of multivalent ligands 32 (approximately 5 µM in the case of 10-12) ConA sites become saturated (35), disassembling clusters and inhibiting cell aggregation.

Figure 10: Bar graph illustrating that ConA clusters assembled on ROMP-derived scaffolds are able to form aggregates of Jurkat cells. Percent of Jurkat cells present in aggregates is plotted against the treatment. ConA at 100 µg/mL or 5 µg/mL is able to form aggregates. Aggregate formation could be inhibited by addition of 50 mM methyl ∀-D-mannopyranoside (∀ man). Compounds 9-12 were added to a final mannose concentrations of 0.5 µM or 5 µM along with a final ConA concentration of 5 µg/mL. Results are the average of at least three independent experiments and error bars represent single standard deviations.

Figure 11: Controlling ConA-mediated erthyrocyte agglutination. A graph of macroscopic aggregation index (%MAI) as a function of time after contact with cells (sec) for treatments with ConA alone or ConA in combination with ligand compound 13 (Scheme 1, mannose containing ligand with n = 100). The concentration of Con A used was 5µg/mL (53 nM, based on ConA tetramer) and ligand (530 nM, based on saccharide). Thus, the ratio of mannose (in the ligand) to ConA tetramer in the experiment was 10:1. Addition of the multivalent ligand significantly enhanced erythrocyte agglutination.

Figure 12: Enhancement of Cell Toxicity of ConA by a Multivalent Ligand. A bar graph indicating % cell viability of PC12 cells as a function of various treatments. "HBS"is the medium control;"ConA"is treatment with 0.1µM ConA (based on Con A tetramer) in HBS medium; "Compound 11"is treatment with 4µM compound 11 (concentration based on saccharide) in HBS; "ConA + Compound 11"is treatment with 0.1µM ConA and 4µM compound 11 in HBS. Addition of the multivalent ligand which binds to ConA significantly enhances ConA toxicity.

### Detailed Description

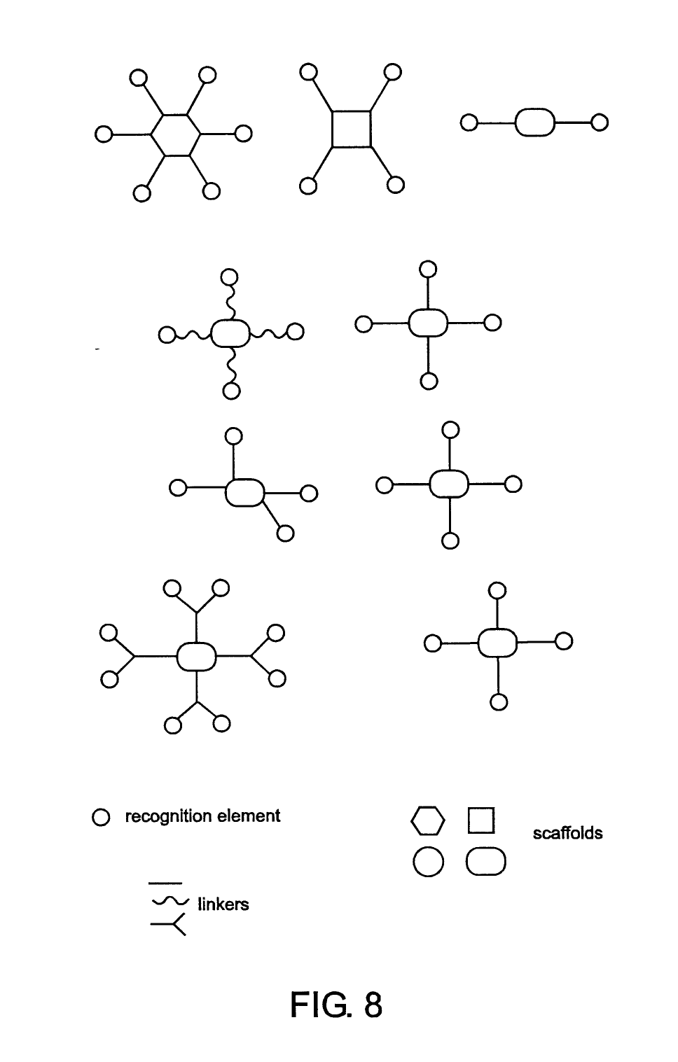

The multivalent ligands of this invention are molecular scaffolds to which a plurality of functional or structural groups, particularly RE and/or SREs, are bonded, to present a display of the functional or structural groups in a productive manner. The scaffold can in general be formed from any chemical or biological species that provides the desired orientation of display. In addition to linear arrays, the scaffolds can be chosen to provide arrays of functional groups with selected non-linear presentation. See, for example, the various non-linear scaffold structures illustrated in Fig. 8.

The functional or structural groups may be bonded to the scaffold in a symmetric or unsymmetric array. The scaffold may comprise a relatively small organic molecule, such as an aromatic ring system (including benzene, naphthalene and fused and non-fused aromatics). Various fused aromatic systems can provide a wide range of different display orientations with functional groups bonded at selected positions on the ring system. Alternatively, saturated ring systems (e.g., cyclohexanes), heterocycles (e.g., carbohydrates), or alicyclic compounds (e.g., tris(hydroxymethyl)aminomethane) can also be used. Molecular scaffolds more typically comprise a plurality of repeating units or monomers, e.g., are polymers or oligomers. The molecular scaffold then carries a plurality of functional or structural groups bonded to repeating units or monomers. The functional groups are bonded covalently or noncovalently to the scaffold and can comprise a plurality of recognition elements (RE), or signal recognition elements (SRE), and can optionally comprise other functional elements (FE).

The RE, SRE and any FE can be bonded on to the molecular scaffold randomly or to a pre-selected pattern in which the arraignment of the RE, SRE and FE along the length of the scaffold matches a selected pattern, e.g., alternating different SRE or RE, selected spacing of different SRE or RE and the like.

The molecular scaffold can be rigid or flexible, hydrophilic or hydrophobic, symmetrical or unsymmetrical, have large surface area or small surface area, and interact or not with cell surface receptors. The molecular scaffold can be any of a variety of oligomers or polymers, including without limitation, polyacrylamides, polyesters, polyethers, polymethacrylates, polyols, and polyamino acids and corresponding oligomers. Molecular scaffolds can in general be linear polymers, branched polymers or cross-linked polymers. Preferred molecular scaffolds are biocompatible. Molecular scaffolds prepared by ROMP methods, as illustrated in several formulas herein, are preferred. Molecular scaffolds can be hydrophobic or can be made to be more hydrophilic by substitution with polar substituents, such as -OH. The scaffold can be substituted, in general, with any groups that do not interfere with signal activity and which provide desirable chemical and physical properties.

The term "recognition element"or RE is used herein to refer to chemical or biochemical species, groups or structures that function for binding to cell receptors and in particular function or binding to cell surface receptors. RE are bonded to molecular scaffolds in the multivalent ligands of this invention. An RE can be a ligand for a cell receptor or a portion of such a ligand that is functional for receptor binding and that has been modified to allow its bonding to a molecular scaffold. An RE can be chemically identical to a cell receptor ligand or it may be modified from the ligand as a result of or to facilitate bonding to the scaffold.

The term "signal recognition element"or SRE is used herein to refer to chemical or biochemical species, groups or structure that function as chemical or biochemical signals (see below) and that are bonded into multivalent ligands of this invention. The SRE is typically a signal (group or molecule) that has been modified to allow its bonding into the multivalent ligand. An SRE can be chemically identical to a signal or it may be modified from the signal as a result of or to facilitate bonding to the scaffold. The SRE is preferably bonded into the multivalent ligand such that the signal function of the group is minimally affected. SREs are recognized by cells, typically by binding to a cell receptor, and thus are also REs. SREs, in contrast to REs, induce a response in or from the cell. The response may be an intracellular response, such as cell migration, and/or an intercellular response, such as the release of chemical species by the cell that function as chemical signals for other cells. Signal recognition is mediated by the presence of cell receptors on the cell surface to which the signal (or signal group) binds. Binding of signal (or SRE) alone may induce the biological response. Induction of the response may in some cases require presentation of multiple signals or (SRE). The biological response may in some cases be modulated by reorganization of receptors or clustering of receptors or the cell surface.

The term "chemical or biochemical signal"is used herein to refer to a particular chemical or biochemical species selected from various types (molecules, oligomers, moieties, groups etc.) that are recognized by a cell most typically by interaction with a cell surface receptor, and induce a biological response in the cell. A signal itself can induce the response on interaction with the cell or may only induce the response when multiple signals interact (e.g., when presented multivalently) with the cell. Signals can include the natural signals, which are those species found in vivo in a biological system to induce a response in or by a cell. Natural signals include, for example, naturally-occurring drugs, hormones, antigens, grow factors, cytokines, proteins, peptides, derivatized peptides (e.g., sulfated, phosphorylated, acylated, or N-formylated peptides), saccharides, derivatized saccharides (e.g., sulfated, acetylated, sialated), nucleic acids, various cell nutrients, epitopes and various small organic compounds (all of which may not represent mutually exclusive groups). Signals can also include chemical species that are found to mimic the function of natural chemical signals. These signal mimics are typically synthetic and can include, for example, synthetic drugs and various derivatives of naturally-occurring signals (e.g., peptoids and nucleic acid analogs or derivatives). Different cells can, of course, recognize different signals. Different cells may respond to the same or similar signals, with the same or with different biological responses. A single cell may respond to a plurality of different signals to give the same or different biological response. Signals include, for example, chemoattractants and epitopes (antigenic determinants) which are not mutually exclusive groups. SREs bound to multivalent ligands can comprise a chemical or biochemical signal adapted for bonding to a molecular scaffold. SREs can include, among others, chemical and biochemical species that are chemoattractants, epitopes, cytokines, hormones and related substances.

A chemoattractant is a chemical or biological signal toward which a cell migrates. The cell senses increasing concentrations of the chemoattractant and moves toward higher concentrations. Cell sensing mechanisms for chemoattractants are often very sensitive. Alternatively, cells may, in response to other signals, move to lower concentrations of signal. Bacterial cells migrate toward certain nutrients, such as glucose or galactose or amino acids, such as serine. Leukocytes (white blood cells) migrate toward, N-formyl peptides and other derivatized peptides, the activated component of CS (CSa), platelet-activating factor(PAF), leukotriene B4 (LTB4), or chemotactic cytokines (i.e., chemokines, including ∀- and ∃-chemokines) (65). N-formylated peptides are products of bacterial protein synthesis and signal bacterial infection. The receptors for N-formylated peptides may also bind to other derivatized peptides such as N-acyl-peptides. Thus any ligand (which may include species that act as agonist or antagonists of receptor function) of a N-formylated peptide receptor may be employed for applications related to that receptor. Neutrophils, one type of leukocyte, are guided to the site of bacterial infection by sensing low levels of N-formylated peptides. Once at the site of infection phagocytosis can occur. A chemoattractant may induce biological responses in addition to migration or chemotaxis. For example, in various types of leukocytes, chemoattractants can induce the release of toxic species or the release of inflammatory cytokines, transcription factors and other chemical species which, in turn, function as chemical signals for other cells.

The term epitope is used generally herein to refer to any chemical species that functions as an antigenic determinant and most generally includes all antigens. Epitopes are those parts of an antigen that combine with an antigen-binding site on an antibody molecule or on a lymphocyte (e.g., B cells and T cells) receptor. Binding of the epitope can, for example, stimulate antibody production or T cell responses. Epitopes may exhibit different levels of immunogenicity. Those that are more immunogenic than others and which dominant the overall antigenic response are designated immunodominant epitopes. Most non-self proteins and many carbohydrates are antigens, so epitopes include, without limitation, proteins fragments (e.g., peptides) and carbohydrate fragments (e.g., saccharides and oligosaccharides). As used herein the term "self"as applied to antigen, epitope or cell is an entity that is recognized by an immune cell, a combination of immune cells or an immune system as self. The term "self" may also be applied other biological particles that are recognized as self by an immune cell, or cells or an immune system. Some antigens, epitopes, cells and particles that are recognized as self are actually foreign to the immune cell, cells or immune system, but are not so recognized. As used herein the term "foreign" as applied to antigen, epitope or cell is an entity that is recognized by an immune cell, a combination of immune cells or an immune system as foreign. Foreign is also any thing that is not recognized as self, i.e., non-self antigens, etc. The term "foreign"may also be applied to other biological particles that are recognized as foreign by an immune cell, or cells or an immune system. Some antigens, epitopes, cells and particles that are recognized as foreign are actually self to the immune cell, cells or immune system, but are not so recognized.

The term hapten takes its generally accepted meaning in the art as a small molecule, having at least one of the determinant groups of an antigen, that can combine with an antibody but is not immunogenic unless it acts in conjunction with a carrier molecule. Haptens include, among others, hemocyanins and nitro-substituted aromatic compounds, such as dinitrophenyl groups, trinitrobenzene sulphonyl groups, and dinitrofluorophenyl groups.

The term antibody as used herein is intended to encompass any protein or protein fragments that function as an antibody and is specifically intended to include antibody fragments including, among others, Fab fragments.

A lectin is any of a large group of hemagglutinating proteins found principally in plant seeds. Certain lectins cause agglutination of erythrocytes of certain blood groups; others stimulate the proliferation of lymphocytes.

The term "biological system"is used generally herein to refer to any in vivo or in vitro system containing signal transduction elements, e.g., signal receptors and biochemical/biological elements for generating a response. A biological system typically contains at least one cell within any environment with which it interacts. A biological system in the context of the uses of multivalent ligands of this invention must contain at least one receptor which can interact with the ligand. In most applications of multivalent ligands, the biological system must contain at least one cell which can respond to the ligand. The response of a cell to the ligand occurs within the biological system and as noted above may be an intracellular response, an intercellular response or both. The biological system can, for example, be a cell in a tissue, a cell in an organ or organism, a cell in a mixture of cells, a cell in a tissue culture, a cell in a tissue or biological fluid sample, and can include biological systems in vivo and in vitro.

"Functional elements (FE)"are chemical or biochemical species (molecules, groups, moieties, etc.) that exhibit some biological or chemical function different from an RE or SRE. FE can, for example, provide reactive groups or latent reactive groups for attaching another chemical or biological group to a multivalent ligand. For example, an FE can be used to attach a multivalent ligand to a solid surface which may be useful for ligand purification or in applications to analytical or diagnostic assays. FE can be various detectable labels or reporter groups including fluorescent labels, radiolabels and high density labels such as gold particles bound to ligands (e.g., streptavidin labeled with gold particles). Multivalent ligands incorporating detectable labels or reporter groups can be used, for example, in various analytical or diagnostic assays. Of particular interest are multivalent ligands of this invention that are useful in visualization assays, e.g., for the detection of biological particles or molecules in microscopy applications. FE can also exhibit various biological functions, e.g., enzymatic function, ligand-binding function, etc., which may facilitate or enhance a selected application of a multivalent ligand.

Attachment of RE, SRE and/or FE can be facilitated by use of linker groups intervening between the molecular scaffold of the multivalent ligand and the signal group. Linker groups can be linear or branched, rigid or flexible, hydrophilic or hydrophobic as desired. One of ordinary skill in the art can select linkers from a variety of chemical species suitable for a given application. Further, one of ordinary skill in the art in view of methods and materials that are well known in the art can readily prepare multivalent ligands with linkers having desirable properties.

Multivalent ligands of this invention can be used to modulate signal transduction in prokaryotic and eukaryotic organisms. The ligands function in a variety of signal transduction processes. Prokaryotes have a highly conserved intracellular signal transduction system, the two component system. The major components of this system are varying numbers of alternating histidine-aspartic acid kinase-mediated phosphorylation events, such as virulence, antibiotic resistance, response to environmental stress and sensing. The components of the two component system are highly conserved in prokaryotes. In contrast, eukaryotes appear to have very few two component systems for signal transduction. This orthogonality makes the two component signaling pathway a prime target for exploitation in therapeutic design for the control of bacterial infection. Major signal transduction systems in eukaryotes are mediated by G-protein-linked receptors and enzyme-linked receptors (including receptor guanylyl cyclases, receptor tyrosine kinases, tyrosine-kinase-associated receptors, receptor tyrosine phosophatases, and receptor serine/threonine kinases). The ability to modulate or regulate signal transduction in these pathways allows control over a wide variety of biological processes in eukaryotic cells and eukaryotic organisms (including mammals and specifically humans) and provides significant opportunity for the design of therapeutics.

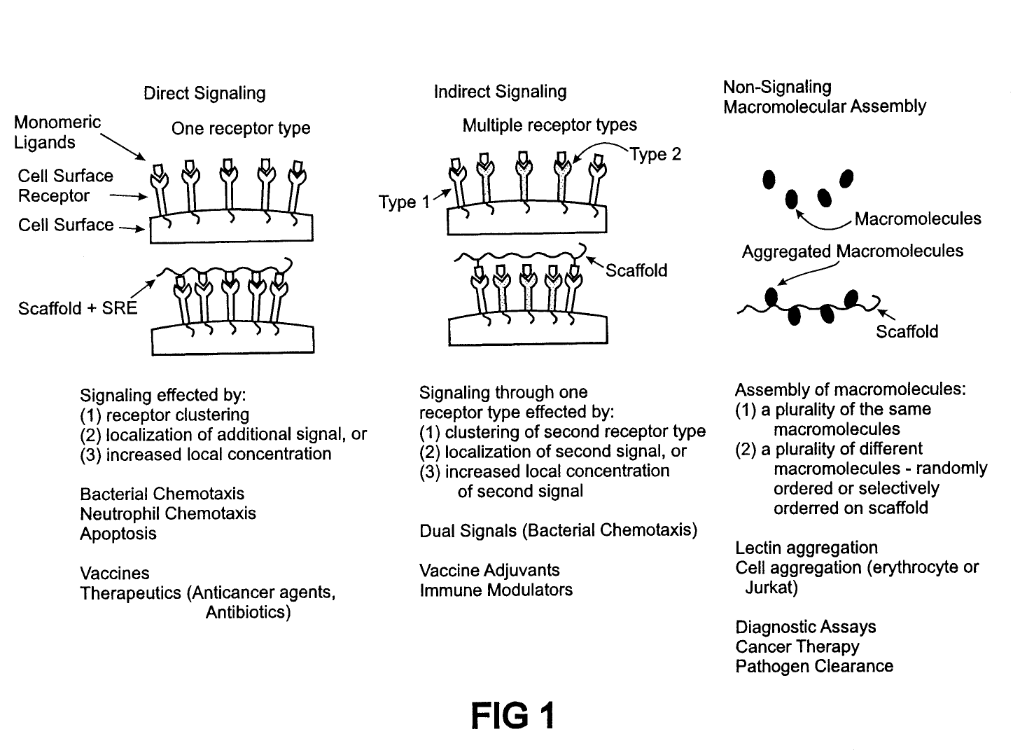

Figure 1 illustrates several mechanisms by which multivalent ligands of this invention can function as effectors of biological response. A multivalent ligand can be involved directly in signaling where SREs on the multivalent ligand bind to cell surface receptors, similar to monomeric ligands, and directly induce (or inhibit) a response. Use of a multivalent ligand of this invention with SRE attached to a molecular scaffold can facilitate receptor clustering or relocalization on the cell surface, localization of second messengers or simply generally increase the affinity by local increase in SRE (ligand) concentration. Multivalent ligands functioning through direct signaling can be employed in a variety of applications, including those based on disruption of biofilm formation or disruption of cell migration, are of particular interest for vaccines, and other therapeutics (cancer treatment and antibiotics).

Multivalent ligands of this invention can also be involved indirectly in signaling (see Fig. 1) affecting the response of a cell to another signal or ligand. Multivalent ligands may function to sensitize or prime cells for enhanced response to another ligand. Indirect signaling effects may be mediated by clustering or reorganization of one type of cell surface receptor which effectively results in the localization or reorganization of other types of cell surface receptors. Multivalent ligands functioning through indirect signaling can also be useful in a variety of applications, particularly those based on enhancement of a biological response, and are of particular interest for vaccines adjuvants and modulators of immune responses.

Multivalent ligands of this invention also have application simply in binding to or targeting of cells. A multivalent ligand containing at least one recognition element for binding to a cell surface receptor (RE) and containing a functional element (FE) targets the cell with that FE. If FE is a label or reporter group, the multivalent ligand acts to label the cell. If FE has a biological function, the multivalent ligand targets the cell with that function.

Multivalent ligands that contain a plurality of RE (SRE or both) can function in macromolecular assembly which need not involve any biological signaling function. In such applications, the multivalent ligand need not contain any SRE, the multivalent ligand need only contain more than one recognition element for binding to a cell surface receptor (a recognition element, RE) and preferably a plurality of REs. In such applications, the multivalent ligands directly or indirectly bind to more than one cell resulting in cell aggregation. Cell aggregation may itself trigger a biological response (e.g., the release of signal molecules by a cell), but need not. Multivalent ligands can indirectly cause cell aggregation by binding to a plurality of biochemical species, such as lectins (e.g., Concanavalin A) which in turn bind to cells resulting in cell aggregation. The effect of a multivalent ligand on indirect cell aggregation will be dependent upon the valency of the ligand and on the relative concentrations of the multivalent ligand to the species that causes cell aggregation. At higher concentrations of multivalent ligands with higher valency, binding sites on the species that causes cell aggregation may be saturated inhibiting cell aggregation. At lower concentrations of multivalent ligand, free binding sites will remain and cell aggregation can occur and can be enhanced by the multivalent ligand. Thus, multivalent ligands of this invention can be selectively designed to inhibit or to facilitate cell aggregation. Multivalent ligands functioning for macromolecular assembly can be useful in a variety of applications, particularly those based on cell aggregation, including, but not limited to diagnostic assays, cancer therapy, and pathogen clearance.

The reorganization of receptors on cell surfaces is involved in many important biological reactions including cell migration, adhesion, and the formation of cell to cell junctions. Multivalent ligands of this invention and in particular those ligands which can span the distance between receptors, as discussed above, can be used to reorganize receptors and to modulate response due to the individual signal interactions with the receptors. Reorganization of receptors on the cell surface includes without limitation: changing the relative positions of different cell receptors on the surface, lateral movement of receptors on the surface, the localization of receptors to different sites on the cell surface, changes in the proximity of signal transduction machinery associated with receptors, changes in the proximity of features of the intracellular matrix associated with receptors, changes in the proximity of receptors, clustering of receptors, changes in conformation of receptors, and initiation of receptor-receptor interactions.

In specific embodiments, linear multivalent ligands of this invention are prepared by ring opening metathesis polymerization (ROMP), see for example (54). This method has been used to prepare multivalent inhibitors of cell functions (27, 28). The ROMP methods have been described in more detail in U.S. patent 5,587,442 relating to multivalent ligands that are polyglycomers. Improvement of ROMP methods for generating block polymers (and oligomers) and for introducing end-groups on ROMP polymers ( and oligomers) have been described in U.S. patent applications 09/335,420 and 09/336,121, both filed June 17, 1999. (These U.S. patent documents are incorporated by reference herein in their entirety particularly for the description of ROMP methods). Scheme 6 illustrates exemplary methods for modification of ROMP backbones, which can be applied in combination with synthetic methods described in the above listed patents and patent applications to synthesize multivalent ligands of this invention. Scheme 6 illustrates a diimide reduction (23, 98, 99) which can be employed to reduce double bonds in ROMP scaffold backbones. Scheme 6 also illustrates the substitution of ROMP scaffold backbones with OH groups using OsO₄ catalyzed dihydroxylation (100, 101). Those of ordinary skill in the art can prepare multivalent ligands of this invention, particularly those specifically exemplified in formulas herein, employing the descriptions herein and methods that are well known in the art.

Multivalent ligands of this invention prepared by ROMP are exemplified by the general structure:

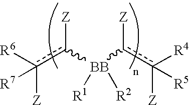

wherein:n is an integer that is 2 or more and represents the number of repeating units in parentheses that are in the ligand;the dashed lines indicate optional double bonds; "BB" represents the backbone repeating unit, which may be cyclic or acyclic, and may be the same or different in a random or block arrangement where the wavy lines indicate that the BB repeating unit can be in a cis or trans configuration in the backbone;R¹ and R², can be H, an organic group, an FE group or the groups:-L-RE- or -L-SRE- wherein FE is a functional element other than an RE or an SRE, L represents an optional linker group, RE is a recognition element and SRE is a signal recognition element;R⁴ and R⁵ are H, or an organic group;R⁶ and R⁷ are H, an organic group or an end-group;Z , independently of other Z in the polymer, is H, OH, OR⁸, SH, a halide (F, Br, Cl, I), NH₂ or N(R⁸ )₂ where R⁸ is H or an organic group or Z is absent when there is a double bond at the carbon to which A is attached.

The integer n is the average number of repeating units in the polymer. Typically n can range up to about 10,000, but there is no practical limit. Preferably the number of repeating units in the multivalent ligands of this invention is defined and can range generally from 2 up to several hundred or several thousand. Preferred multivalent ligands will have n that ranges from 10 to about 100. Multivalent ligands of this invention also include those in which n ranges from 10 to about 25, in which n is 25 or more and those in which n is 50 or more. ROMP can provide polymers of varying average lengths (i.e, varying degree of polymerization, DP) depending on the monomer to ROMP catalyst (i.e., initiator ) ratios. The length of all polymers referred to herein (i.e., n or n+m, below) is the length predicted by the monomer to initiator ratios used in the polymerization reaction.

BB can be alkyl, cycloalkyl, cycloalkenyl, and one or more CH₂ groups in the BB moiety can be replaced with -O-, -S-, -NR⁹-, or -CO-, where R⁹ is H or an organic group. Preferred BB have 10 or fewer carbon atoms.

Exemplary BB repeating units include among others:

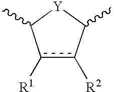

RE is a recognition element as discussed above that can be any of a variety of chemical or biochemical species that are recognized by and which selectively bind to cell receptors, particularly, transmembrane receptors and cell surface receptors. SRE is a signal recognition element as discussed above that can be any of a variety of chemical or biochemical species that are recognized by one or more cells and which induce a biological response by the cell; "L" is an optional linker group that can provide functional groups for covalent bonding of the RE, SRE or FE to the polymer (oligomer) backbone. FE is a chemical or biochemical functional group other than an SRE, as discussed above. Other examples of ROMP scaffolds are illustrated in Schemes 2 and 3.

The multivalent ligand of the above formula contains up to n RE, SRE or both. In specific embodiments all of the monomers carry an RE or SRE (the number of RE + SRE is n). In other specific embodiments, regions of spacer monomers that do not carry RE or SRE intervene between regions of monomers that carry RE or SRE. The RE and SRE attached to different monomers may be the same or different. In one embodiment, RE or SRE throughout the multivalent ligand are all the same. In another embodiment, the multivalent ligand contains more than one type of RE or SRE. In a specific embodiment, the multivalent ligand contains two different types of RE or SRE or an RE and an SRE. In this embodiment, the RE and SRE are non-randomly positioned in the ligand. Preferably monomers carrying the same RE or SRE are grouped into blocks (as in block polymers) within the multivalent ligand and spacer monomers are optionally positioned between blocks. In other embodiments, R¹ and R² together can form an RE or SRE.

RE and SRE are attached to the polymer (oligomer) backbone such that they substantially retain their function for binding cell receptors or as signals, respectively. For a given RE or SRE there may be several ways in which it can be bonded into the multivalent ligand, each of which may result in RE that are different in binding affinity or SRE that are different either in binding affinity or in the level or type of response induced. For example, a peptide signal may be bonding through its N-terminus, through its C-terminus or via an amino acid side group, such as through a lysine side group. The site of attachment of an RE or SRE to the multivalent ligand is preferably selected to minimize loss of binding function (RE) or to minimize loss of signal function (SRE) or alternatively the site of attachment may be selected to maximize signal function (SRE). An RE or SRE may nevertheless exhibit properties that are different from free ligands or free signals (e.g., the binding affinity of an SRE for a cell receptor may be different from that of free signal from which is was derived or which it mimics), but which do not destroy the function of an RE as a ligand or an SRE as a signal. RE can include a variety of known cell receptor ligands and in particular can include lectins. SRE can specifically include monosaccharides (e.g., glucose, galactose), disaccharides, polysaccharides (greater than 2 sugar residues), derivatized saccharides (e.g.,acylated, sialated), peptides, derivatized peptides (e.g., N-formyl peptides), peptoids, various chemoattractants, and various epitopes. Note that a particular chemical or biological species may function as an RE with one type or kind of cell and as an SRE with another type or kind of cell.

The linker can provide for spacing of the RE, SRE or FE group(s) from the backbone or can provide for structural flexibility. Linkers may be the same or different on different monomers in the polymer. Linkers that are used in a monomeric scaffold to bond to RE, SRE or FE can also be all the same or different. In a given multivalent ligand carrying one type of RE or SRE group, the linker is preferably the same throughout the polymer. Linkers are generally selected so that they are compatible with the intended application of the multivalent ligand and to avoid interference with the function of signal groups. The linker is preferably linear and preferably ranges in length from 1 to about 20 atoms. The linker may contain alicyclic groups (such as a cyclohexyl group). The linker can be an alkyl chain carrying functional groups for bonding to the backbone of the ligand and to the signal. The linker can also be an ether, ester, ketone, amine, amide or thioether chain. In a specific embodiment, the linker can be described as an linear alkyl chain having from 1 to about 20 carbon atoms in length in which one or more non-neighboring CH₂ groups are optionally replaced with an -O-, -S-, -NH-, -NR¹⁰-, -CO-, -NH-CO- , -O-CO-, -HC=CH- , or -C≡C- group, where R¹⁰ is an alkyl or aryl group. Linker CH₂ groups can be substituted with halogens, alkoxy, or alkyl groups. In the absence of a linker group, the ROMP backbone or the signal group itself must provide the functionality for covalent bonding of the signal to the backbone. Exemplary linkers include those illustrated in Scheme 3.

R¹, R², R⁴, R⁵, R⁶, R⁷, R⁸ and R⁹ can be organic groups. Organic groups include without limitation alkyl groups, alkenyl groups, and aryl groups as well as substituted alkyl, alkenyl and aryl groups. Substituents for alkyl, alkenyl and aryl groups include halogens (F, Cl, Br, I), -CN, -NO₂, -OH, -SH, -NH₂, -N(R¹⁰)₂, -SR¹⁰ and -OR¹⁰ where R¹⁰ is an alkyl or aryl group. Aryl groups may also contain alkyl or alkenyl substituents. Organic groups will typically have from 1 to about 20 carbon atoms, and preferably have 1 to about 10 carbon atoms. Alkyl groups may be straight-chain, branched or cyclic (or contain portions that are cyclic). One or more non-neighboring -CH₂- groups in an alkyl or alkenyl group can be replaced with -O-, -S- , -NH- or -NR¹⁰, where R¹⁰ is an alkyl or aryl group.

R⁶ and R⁷ can be end-groups, such as those described in U.S. patent application 09/336,121 filed June 17, 1999 which is incorporated in its entirety herein for description of methods of synthesis of multivalent ligands having end-groups using ROMP methods. End-groups can include a latent reactive group or a non-reactive functional group as described in the cited patent application. The presence of a latent reactive group would allow for later functionalization of a polymer multivalent ligand at an end-group. End-groups can contain functionality for binding to solid surfaces. The end-group may itself be a linkage to a solid support material. Latent reactive groups include: azides, a nitro group, a disulfide, a cyano group, an acetal group, a ketal, a carbamate, a thiocyanate, an activated ester, or an activated acid (activated esters and acids are those containing good leaving groups that are activated in particular for nucleophilic attack). Non-reactive end-groups include natural products or analogs thereof (e.g., biotin), metal chelators (e.g., nitrilotriacetic acid), metals (e.g., Zn), and fluorescent labels (amide derived BODIPYL FL EDA which is 4,4-difluoro-5,7-dimethyl-4-boro-3a,4a-diaza-s-indacene-3-propionyl ethylenediamine). End-groups can include FE.

The multivalent ligand optionally contains one or more functional elements that are not SRE. Preferred multivalent ligands contain significantly fewer FE compared to SRE. FE can be or contain any of the reactive or non-reactive groups listed above or described in U.S. patent application 09/336,121 filed June 17, 1999 as "end-groups". FE can also have enzymatic or other protein function.

When prepared by the ROMP methods, such as those described in U.S. patent applications 09/335,420 and 09/336,121, both filed June 17, 1999 (which are incorporated by reference herein in their entirety for methods of synthesis of multivalent ligands), R⁴ and R⁵ are derived from the metal carbene catalyts, i.e., they are substituents on the metal carbene carbon of the metal carbene catalyst and in specific embodiments are H and a phenyl group. When using ROMP, R⁶ and R⁷ are typically derived from the capping agent, i.e, are the substituents on the electron-rich alkene capping agent, such as hydrogen in the case of ethyl vinyl ether.

In a specific embodiment multivalent ligands of this invention include those of formula:

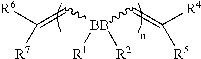

wherein BB, R¹⁻², and R⁴⁻⁷ are as defined above. In specific embodiments, one of R¹ or R² is H and the other is L-RE. In specific embodiments, one or R¹ or R² is H and the other is L-SRE. In specific embodiments, RE is a lectin or a cell receptor ligand that is comprised within a lectin. In specific embodiments, SRE is a monosaccharide, a disaccharide or a relatively short saccharide having up to about 10 sugar residues. In other specific embodiments, SRE is a peptide or a derivatized peptide (e.g., an N-formyl peptide).

In another specific embodiment the invention relates to multivalent ligands of the formula:

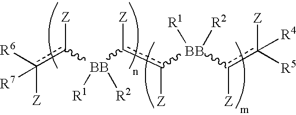

wherein the dashed line indicates an optional double bond and wherein Y, independently of Y in other monomers, R¹⁻², independent of R¹⁻² in other monomers, and R⁴⁻⁷ are as defined above. In specific embodiments, Y is -CH₂-. In specific embodiments, one of R¹ or R² is H and the other of R¹ or R² is -L-RE. In specific embodiments, one of R¹ or R² is H and the other of R¹ or R² is -L-SRE. R¹ and R² together may form an - L-RE or -L-SRE. In yet other specific embodiments, SRE is a peptide or derivatized peptide. When no double bond is present the ring carbons typically carry addition hydrogens, but may be substituted with other groups, such as alkyl groups having 1-6 carbon atoms or halides that do not interfere with the function of any R¹ or R² group.

In yet another specific embodiment the invention relates to multivalent ligands of the formulas:

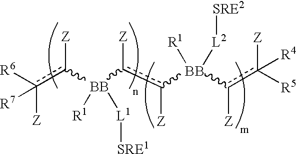

or

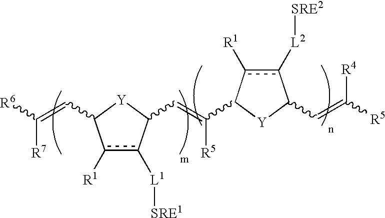

in which m is the number of monomers carrying a first SRE (SRE¹) and n is the number of monomers carrying a second SRE (SRE²). L¹ and L² are linkers as described above which may be the same or different. All other variables are as defined in earlier formulas and dashed lines indicating optional double bonds. Both m and n are integers that can range most generally from 1 up to about 10,000, but which more typically will range from 1 to several hundred or several thousand. The value of m may be the same as or different from that of n. In preferred ligands, n + m ranges from 5 or more up to about 200. Multivalent ligands of this invention include those in which n + m ranges between about 10 and 25, those in which n + m is 25 or more, those in which n + m is 50 or more, and those in which n + m is 100 or more.

Other exemplary multivalent ligands include those of the formulas:

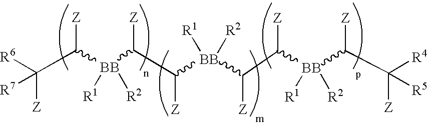

wherein n, m and p are integers with a value greater than 3 and other variables are as defined above and

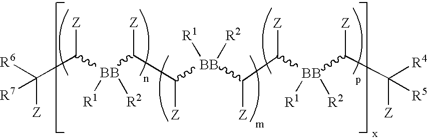

wherein n, m, p and x are integers each of which has a value greater than 1 and all other variables are as defined above. Multivalent ligands of these formulas can contain multiple blocks of monomer regions having the same RE or SRE. Multivalent ligands of these formulas can contain multiple blocks of monomer regions one RE or SRE and multiple blocks of monomer regions containing another RE or SRE. Multivalent ligands of these formulas can also contain multiple blocks of monomer regions carrying RE or SRE with intervening spacer regions that carry no RE or SRE.

The multivalent ligands of this invention are useful in methods for controlling or modulating the effect of chemical signals in a biological system. Applications of multivalent ligands to bacterial and eukaryotic chemotaxis, to migration of leukocytes (particularly neutrophils), to immune responses of B-cells and T-cells, to cell aggregation, and to signaling of apoptosis are exemplified herein below.

Multivalent ligands of this invention which carry bacterial chemoattractants can be employed to disrupt colonization and biofilm formation by bacteria. Chemotaxis is a virulence factor which facilitates bacterial colonization of its host. Disruption of colonization of host tissue prevents host-bacterial interactions, prevents colonization and inhibits or retards infection. The methods of this invention can be applied specifically to disruption of colonization, for example, by Staphylococus aureus (for treatment of staph infections) and Vibrio cholerae (for treatment of cholera). One bacterial survival mechanism involves the formation of microcommunities with functional heterogeneity (biofilms). Biofilm formation and maintenance are regulated by soluble small molecule-based factors. These factors control signal transduction pathways that allow bacteria to sense their environment and conversions to biofilm formation are mediated by two-component signaling systems. Disruption of biofilm formation renders bacteria more susceptible to host defenses or to antibiotic treatment and can inhibit or retard infection. Multivalent ligands which disrupt biofilm formation can be particularly useful in preventing or treating infections of the lung, for example for treating or preventing lung infection by the opportunistic pathogen Pseudomonas aeruginosa. Infection by this organism is a leading cause of death in patients with cystic fibrosis. Another mechanism for bacterial survival is induction of a virulence response upon increased bacterial cell density. This virulence response is induced by the release of soluble factors when increased cell density is sensed. Disruption of the responses of bacteria to increased cell density by multivalent ligands of this invention can be used to control bacterial virulence, for example, this method is applicable to the control of virulence of Staphylococus aureus.

Multivalent ligands of this invention can in similar ways be employed to disrupt infection by eukaryotic pathogens and parasites, including among others, Trypanosoma cruzi (chuga disease) Trypanosoma brucei (sleeping sickness), tapeworms, hookworms, and Plasmodium falciparum (malaria).

The multivalent ligands of this invention can be used to modulate immune response toward epitopes and antigens (e.g. by modulating the immunogenicity of these species). For example, multivalent ligands can be designed to stimulate or inhibit leukocyte responses, including migration. Stimulation of such response can be used to enhance recognition of non-self cells for clearance and treat infection. Multivalent ligands can also be designed to modulate the activation and/or deactivation of B-cells or T-cells in response to chemical signals to improve and enhance desired immune response. B-cells and T-cells can be treated with multivalent ligands of this invention in vitro, in vivo and ex vivo.

Autoimmune diseases involve aberrant function of a cell signal recognition process in which self cells are incorrectly marked for clearance. Multivalent ligands of this invention which modulate cell responses of immune system cells to epitopes can be employed to inhibit or attenuate autoimmune disorders. In a specific embodiment, ligands carrying self epitopes mistakenly recognized as "non-self" and certain B-cell or T-cell epitopes can be employed in a tolerization process to ameliorate autoimmune responses.

The multivalent ligands of this invention also have application to the treatment of undesired cell proliferation (cancer) and undesired cell migration (metastasis). Cancer cells have distinct surface features (e.g., epitopes) that distinguish them from non-cancer cells. The multivalent ligands of this invention can be designed to promote recognition of cancer-specific epitopes as non-self cells by the immune system such that cancer cells are cleared by the immune system. Multivalent ligands carrying cancer cell epitopes and B-cell or T-cell epitopes can be employed in a sensitization process to promote clearance of the cancer cells. Cancer metastasis is deviant cell migration. The movement, adhesion, and junction formation of cancer cells are mediated, at least in part, by interaction of cancer cells with the multivalent extracellular matrix. Multivalent ligands can be designed to inhibit or prevent movement, adhesion and junction formation and thus inhibit metastasis.

This invention provides pharmaceutical and therapeutic compositions comprising multivalent ligands with SRE groups selected to provide therapeutic benefit in combination with a pharmaceutically acceptable carrier or excipient adapted for use in human or veterinary medicine. The multivalent ligands may be combined with each other to achieve a desired pharmaceutical response or administered in combination with other known drugs or therapeutic agents, including without limitation antibacterial and other antimicrobial agents. The multivalent ligand is present in the pharmaceutical compositions in an amount, or in combination with other ligands in a combined amount, sufficient to obtain the desired therapeutic benefit. The carrier or excipient is selected as is known in the art for compatibility with the desired means of administration, for compatibility with the selected multivalent ligand(s) and to minimize detrimental effects to the patient.

This invention is also directed to pharmaceutically acceptable esters and salts of the multivalent ligands of various formulas and structures described herein. Acid addition salts are prepared by contacting compounds having appropriate basic groups therein with an acid whose anion is generally considered suitable for human or animal consumption. Pharmacologically acceptable acid addition salts include but are not limited to the hydrochloride, hydrobromide, hydroiodide, sulfate, phosphate, acetate, propionate, lactate, maleate, malate, succinate, and tartrate salts. All of these salts can be prepared by conventional means by reacting, for example, the selected acid with the selected basic compound. Base addition salts are analogously prepared by contacting compounds having appropriate acidic groups therein with a base whose cation is generally considered to be suitable for human or animal consumption. Pharmacologically acceptable base addition salts, include but are not limited to ammonium, amine and amide salts.

Pharmaceutically acceptable esters of compounds of this invention are prepared by conventional methods, for example by reaction with selected acids. Pharmaceutically acceptable esters include but are not limited to carboxylic acid esters R^{E}COO-D (where D is a cationic form of a compound of this invention and where R^{E} is H, alkyl or aryl groups).

This invention is also directed to prodrugs of multivalent ligands and derivatives which on being metabolized will result in any of the ligands of this invention. Labile substituents may be protected employing conventional and pharmaceutically acceptable protecting groups removable on metabolism. Pharmaceutically active compounds may be derivatized by conventional methods to provide for extended metabolic half-life, to enhance solubility in a given carrier, to provide for or facilitate slow-release or timed-release or enhance or affect other drug delivery properties.

The multivalent ligands according to the invention may be formulated for oral, buccal, parenteral, topical or rectal administration. In particular, the ligands according to the invention may be formulated for injection or for infusion and may be presented in unit dose form in ampules or in multidose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form for constitution with a suitable vehicle, e.g. sterile, pyrogenwater, before use.

The pharmaceutical compositions according to the invention may also contain other active ingredients, such as antimicrobial agents, or preservatives. In general, pharmaceutical compositions of this invention can contain from 0.001(by weight) of one or more of a multivalent ligands described herein.

For administration by injection or infusion, the daily dosage as employed for treatment of an adult human of approximately 70 kg body weight will range from 0.2 mg to 10 mg, preferably 0.5 to 5 mg, which can be administered in 1 to 4 doses, for example, depending on the route of administration and the clinical condition of the patient. These formulations also include formulations in dosage units. This means that the formulations are present in the form of a discrete pharmaceutical unit, for example, as tablets, dragees, capsules, caplets, pills, suppositories or ampules. The active compound content of each unit is a fraction or a multiple of an individual dose. The dosage units can contain, for example, 1, 2, 3 or 4 individual doses for ½, 1/3 or 1/4 of an individual dose. An individual dose preferably contains the amount of active compound which is given in one administration and which usually corresponds to a whole, one half, one third or one quarter of a daily dose. The magnitude of a prophylactic or therapeutic dose of a particular multivalent ligand will, of course, vary with the nature of the severity of the condition to be treated, the particular ligand compound and its route of administration. It will also vary according to the age, weight and response of the individual patient.

The compounds of the present invention are preferably formulated prior to administration. The present pharmaceutical formulations are prepared by known procedures using well-known and readily available ingredients. In making the compositions of the present invention, the active ingredient will usually be mixed with a carrier, or diluted by a carrier, or enclosed within a carrier which may be in the form of a capsule, sachet, paper or other container. When the carrier serves as a diluent, it may be a solid, semior liquid material which acts as a vehicle, excipient or medium for the active ingredient. The compositions can be in the form of tablets, pills, powders, lozenges, sachets, cachets, elixirs, suspensions, emulsions, solutions, syrups, aerosols (as a solid or in a liquid medium), ointments containing for example up to 10% by weight of the active compound, soft and hard gelatin capsules, suppositories, sterile injectable solutions and sterile packaged powders.

Some examples of suitable carriers, excipients, and diluents include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, methyl cellulose, methyl and propylhydroxybenzoates, talc, magnesium stearate and mineral oil. The formulations can additionally include lubricating agents, wetting agents, emulsifying and suspending agents, preserving agents, sweetening agents or flavoring agents. The compositions of the invention may be formulated so as to provide quick, sustained or delayed release of the active ingredient after administration to the patient by employing procedures well known in the art.

The compositions are preferably formulated in a unit dosage form, each dosage containing from about 0.5 to about 150 mg, more usually about 0.1 to about 10 mg, of the active ingredient. The term "unit dosage form" refers to physically discrete units suitable as unitary dosages for human subjects and other mammals, each unit containing a predetermined quantity of active material calculated to produce the desired therapeutic effect, in association with a suitable pharmaceutical carrier.

The invention is further directed to therapeutic methods that comprise the step of administering a pharmaceutical composition of this invention to an individual that can derive therapeutic benefit from the compositions.

Multivalent ligands of this invention can also be employed in non-therapeutic applications, for example, to prevent or inhibit biofouling in a selected environment or to remove undesired cells from a selected environment. Compositions of this invention for use in such non-therapeutic comprise one or more multivalent ligands of this invention in an amount or in a combined amount effective for obtained a desired function, e.g., effective for affecting bacterial or microbial chemotaxis or effective for aggregating cells in a sample or a biological system. Compositions can be formulated using any appropriate solvent or carrier system which may be an aqueous solution, a lyophilized or a spray-dried material so long as desired function is maintained.

The following examples, further illustrate and further describe the invention, but are in no way intended to limit the invention.

THE EXAMPLES

Modulation of Bacterial ChemotaxisThe molecular events leading to bacterial chemotaxis have been well studied, and the process has served as a general model for receptor-mediated responses [29-32]. During chemotaxis in Escherichia coli, chemoattractants, such as sugars and amino acids, and chemorepellents are recognized by specific receptors at the bacterial plasma membrane [33]. For these investigations of multivalent ligand activity, galactose was selected as a model chemoattractant. The related compound, ∃-methyl galactopyranoside, is also a chemoattractant, indicating that the attachment of substituents at the anomeric position of galactose does not abolish its chemotactic activity [34]. This observation suggests that galactose residues can be tethered through an anomeric linker to create a multivalent display. For galactose-mediated signaling, the saccharide must bind to the soluble periplasmic glucose/galactose-binding protein (GGBP), which, in turn, interacts with the galactose-sensing chemoreceptor, Trg [34, 35]. Galactose-GGBP binding to Trg initiates a signaling pathway that results in reversal of the direction of flagellar spin, allowing the bacteria to swim towards the nutrient [29, 30, 36].

Bacterial chemotaxis requires an extremely sensitive sensing system with a broad dynamic range. Through their chemoreceptors, bacteria can detect very small changes in ligand concentration over many orders of magnitude [37, 38]. A recent mathematical model proposed by Bray et al. to explain this remarkable feature suggests signal transduction is regulated by changes in lateral clustering of the chemoreceptors [39-41]. In this model, clusters of bacterial chemoreceptors exchange ligand binding information, such that receptor clusters are more active in signal generation than individual receptors [39, 41]. Multivalent ligands of this invention having distinct valencies can differentially reorganize the receptors and thus control lateral receptor organization may result in modulation of the chemotactic response.

Galactose-bearing ligands 1-4 of varying defined valencies were generated using ROMP methods (Scheme 1). The galactose residues in the multivalent ligands are tethered to the molecular scaffold (polymer backbone) via a short linker. The interaction of monomer 1 was at least as favorable as that of galactose in an in vitro binding assay, thus the attachment of the linker did not prevent galactose binding to purified GGBP.

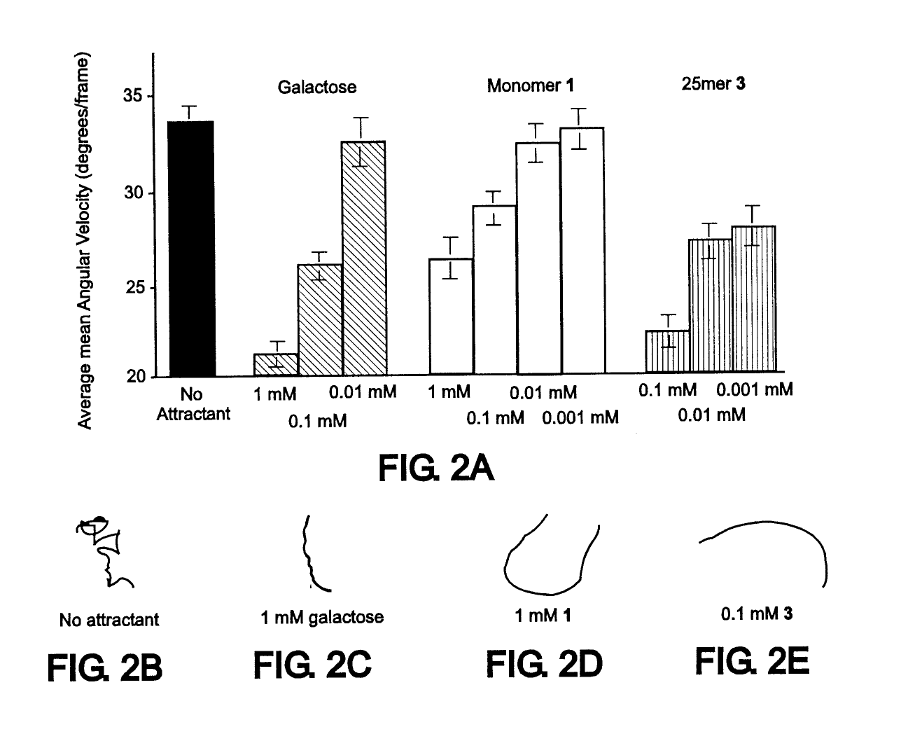

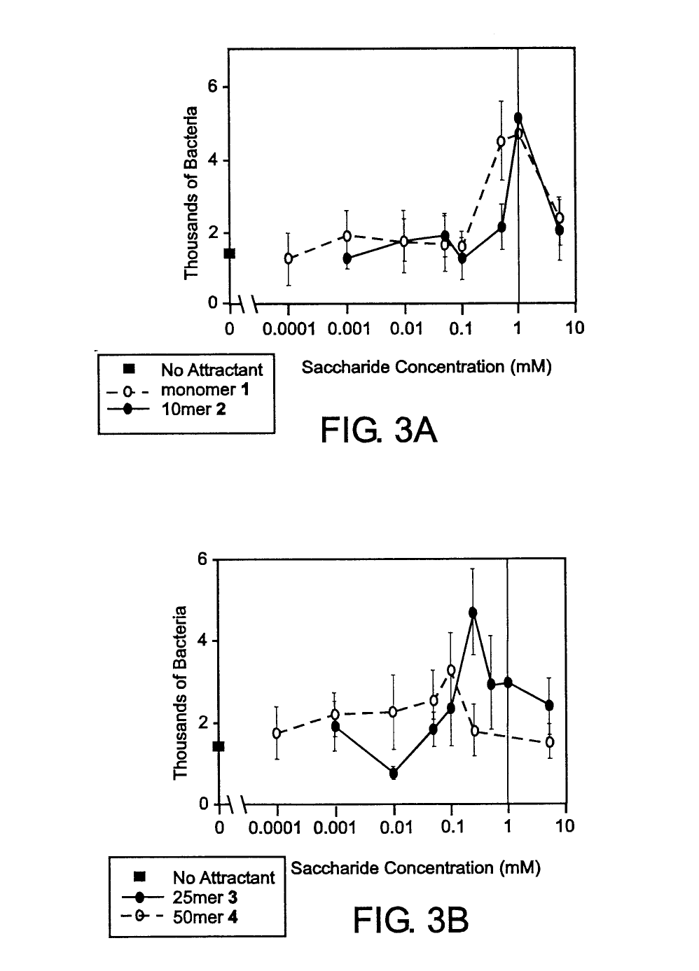

Ligands functionalized with galactose such as monovalent ligand 1 and multivalent ligand 3, also serve as attractants in vivo. This was demonstrated by monitoring the behavioral response of E. coli to these ligands. The locomotion behavior of E. coli occurs in two modes, running and tumbling, which are defined by the direction of the flagellar spin and, ultimately, the signal transduction response that arises from interaction of chemoreceptor with ligand [42]. Bacteria in the presence of an attractant will undergo prolonged running responses with low tumbling frequency [42, 43]. To observe the effects of synthetic ligands on tumbling frequency, E. coli were treated with galactose or galactose-bearing ligands, and bacterial motion was recorded and analyzed using the method of Sager et al. [44]. The tumbling frequency was assessed by averaging the mean angular velocity of the paths obtained in the first 5-15 seconds after addition of attractant (Figure 2A). When bacteria were treated with increasing concentrations of galactose, the mean angular velocity decreased, indicative of a running response. Figures 2B-E illustrate sample paths for representative bacteria treated with buffer alone, galactose, compound 1 and compound 3. Treatment with monovalent compound 1 produced similar effects to that of the free chemoattractant (galactose), indicating that the anomeric substituent in 1 did not preclude chemotactic activity. Multivalent compound 3 was more active than monovalent 1 or unmodified galactose. Multivalent compound 3 induced a low mean angular velocity even at very low (e.g., 0.001 mM) saccharide residue concentrations. The response of the bacteria to 3 at 0.1 mM saccharide residue concentrations (ca. 0.004 mM concentration) was comparable to that obtained at ten fold higher (1 mM) concentrations of unmodified galactose. The observed differences in concentration of maximum activity between the monomer 1 and multivalent compound 3 demonstrate that ligand valency affects chemotactic activity.

E. coli were subjected to concentration gradients of compounds 1-4 in capillary accumulation assays [45] to determine the concentration at which the maximum chemotactic response is achieved and the number of bacteria that accumulate at this maximum [34].

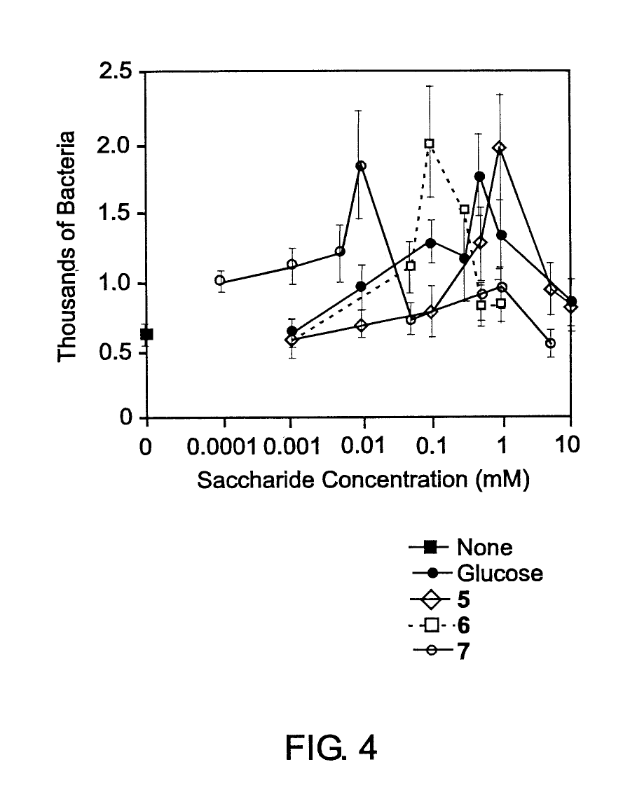

When compounds 1-4 were used as attractants in the capillary accumulation assay, oligomer 2 was no more active than monovalent 1; both elicited a maximum chemotactic response at 1 mM (Figure 3A). Compound 2 displays a higher local concentration of galactose to the receptor, however, the similarity of activities for 1 and 2 indicates that a high local concentration of attractant does not alone give rise to increased chemotactic activity. For compounds 3 and 4, concentrations of maximum chemotaxis were significantly lower; the maximum for 3 is at a galactose residue concentration of 0.25 mM (ca. 0.01 mM ligand concentration, 100-fold lower than free galactose) and the maximum for 4 is at a galactose residue concentration of 0.10 mM (0.0002 mM ligand concentration). Concentrations of maximum chemotaxis of 3 and 4 are 100- and 5000-fold lower, respectively, than free galactose (Figure 3B). The ligands of higher valency (3 and 4), therefore, can induce chemotaxis at extremely low concentrations.

Chemotaxis receptors have been found to be approximately 90 Åapart [46]. Molecular modeling studies indicate that the maximum length of oligomer 2 is approximately 50 Å[23]. The significantly higher potency of the longer oligomers 3 and 4, compared to that of oligomer 2, is believed to be due to the ability of the longer oligomers to cluster chemotaxis receptors. Compound 1 was not a chemoattractant for ggbp (AW550 and AW543) or trg (AW701) E. coli mutants. The results obtained indicated that the ligands 1- 4 act specifically to affect chemotaxis through the galactose-sensing machinery.

The number of E. coli accumulated in assays employing 1-4 (see Figures 3A and 3B) is less than that when galactose is used as an attractant (120,000 bacteria [34]), despite the observed potency of these ligands in the video assays (see Figure 2A). Capillary accumulation assays depend on proper bacterial reorientations to travel into the capillary for collection. The potency of these ligands may disrupt the ability of bacteria to reorient, decreasing the apparent number of bacteria accumulated. At a given saccharide residue concentration of a multivalent ligand, fewer molecules are present to activate the receptors, and these molecules must traverse the outer membrane. These features of the system may also contribute to the decreased numbers of bacteria accumulated.

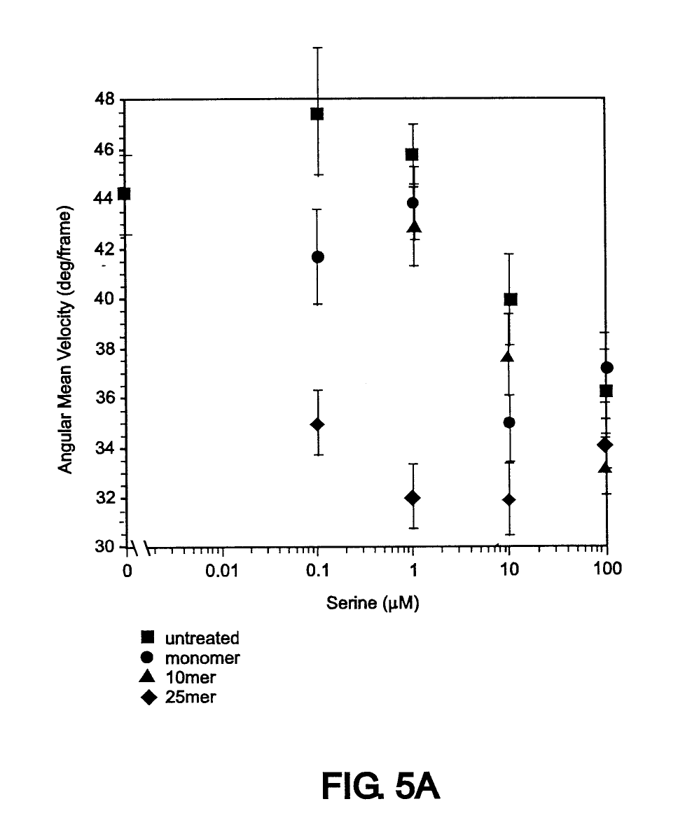

To test the generality of the observed valency-dependent differences in chemotactic activities and to investigate the role of membrane permeability in responses to our ligands, chemotactic experiments in B. subtilis were conducted. B. subtilis is a gram-positive bacterium that, like gram-negative E. coli, is able to respond to saccharide chemoattractants [47, 48]. In the case of B. subtilis, the multivalent ligands can directly interact with saccharide-sensing receptors, without having to first traverse the outer membrane. Glucose is a chemoattractant for B. subtilis [47], but galactose is not. Glucose-carrying ligands (compounds 5-7, Scheme 1) were effective chemoattractants for B. subtilis as shown in capillary accumulation assays. In addition the chemotactic responses to glucose-carrying ligands were shown to also depend on ligand valency. As shown in Fig. 4, monomer 5 elicited maximum activity at 1 mM, while oligomer 6 elicited maximum activity at a saccharide residue concentration of 0.1 mM (50-fold lower ligand concentration than that of free glucose) and oligomer 7 elicited maximum activity at a saccharide residue concentration of 0.01 mM (1250-fold lower ligand concentration than that of glucose). Free chemoattractant signal glucose had maximal activity as a chemoattractant at 0.5 mM. In analogy to observations with E. coli, as the valency of the ligand increases, the saccharide residue concentration of maximum chemotaxis decreases. Significantly, the number of bacteria accumulated towards 5-7 was comparable to the number accumulated when unmodified glucose was used as the attractant. Consistent with previous reports on the activity of galactose, galactose-bearing ligands (such as 1) were not chemoattractants for B. subtilis [47], further indicating that the multivalent ligands were acting specifically. The results observed indicate that in evolutionarily divergent bacteria E. coli and B. subtilis, the valency of the attractant influences chemotactic response.

Fluorescence microscopy experiments were performed to visualize changes in chemotaxis receptor organization upon treatment with saccharide-carrying ligands. These experiments can determine directly whether or not multivalent ligands can influence chemoreceptor reorganization. It had been shown that wild-type E. coli localize chemoreceptors to their poles and that inactivation of the structural protein, CheW, results in a random distribution of chemoreceptors on the cell [49]. The ability of ROMP-derived arrays to localize the chemoreceptors was examined using E. coli cheW mutants. Bacteria were treated with 1, 3, or 4, fixed, and labeled with an antibody to the bacterial chemoreceptors (anti-Tsr). Monovalent compound 1 had no effect on receptor distribution, but multivalent compounds 3 and 4 were observed to reorganize the chemoreceptors. As anticipated, localized receptors in the cheW cells occurred at seemingly random locations, in contrast to the polar localization observed in the wild type bacteria. Receptor clustering was more pronounced in the case of cells treated with the longer oligomer 4 than with 3. The results of these experiments indicate that ROMP-derived multivalent compounds can induce lateral receptor reorganization. The differences observed in chemotactic activities of the multivalent ligands as a function of oligomer length and the observation that ROMP-derived multivalent ligands can induce lateral receptor reorganization supports the conclusion that receptor reorganization is involved in mediating the chemotaxis response. The results further indicate that changes in receptor localization can give rise to changes in chemotactic responses.

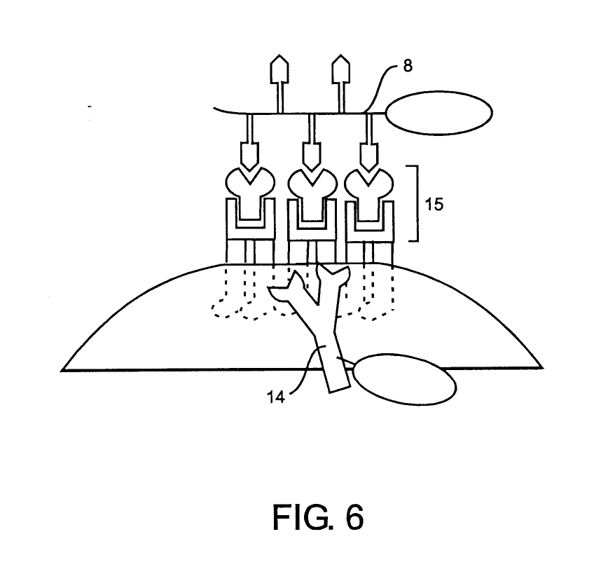

To confirm the ability of multivalent ligands to alter the organization of the chemoreceptors in the bacterial membrane E. coli were treated with compound 8, a galactose-bearing multivalent ligand having a fluorescent label (Scheme 1). When E. coli were treated with 8 or a fluorescein-labeled anti-Tsr antibody 14, the fluorescence patterns observed were similar. Both materials were observed to bind at the poles of the bacteria indicating that the ROMP-derived ligands bind specifically to the bacterial chemoreceptors. To address directly the ability of these multivalent ligands to reorganize receptors 15, CheW mutants were treated with both compounds. Patches of anti-Tsr antibody labeled chemoreceptors that colocalize with compound 8 were observed, as illustrated in Fig. 6. This result indicates that multivalent ligand 8 is responsible for the observed changes in cell receptor organization.

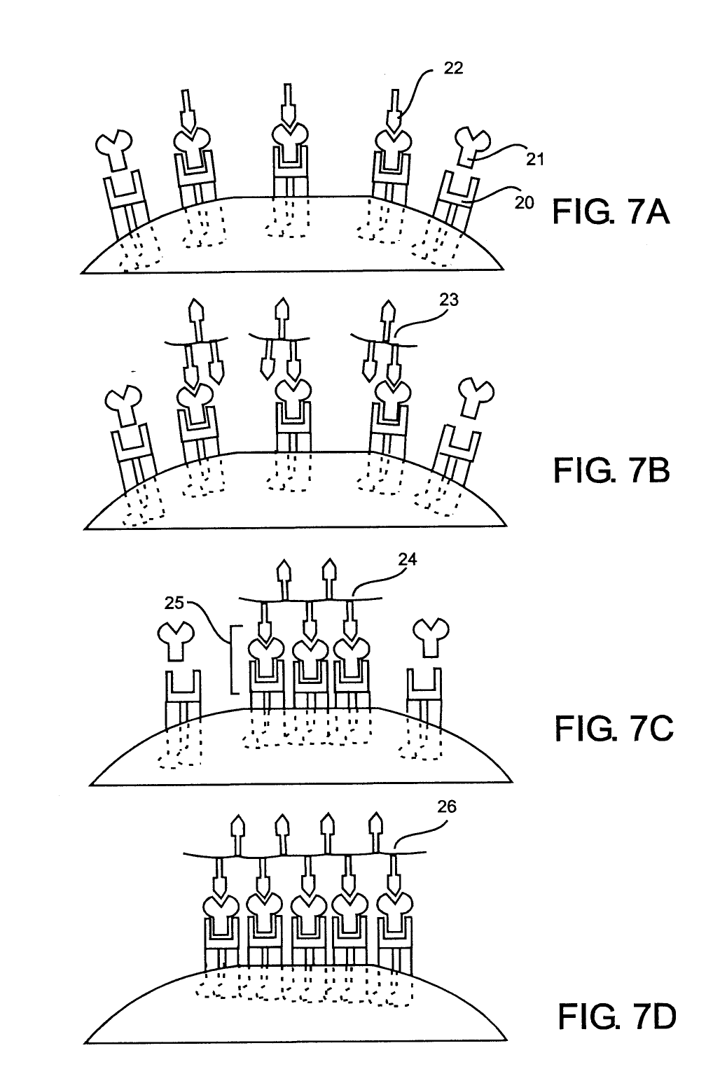

The data obtained indicate that multivalent ligands influence chemotactic responses by altering the organization of cell surface chemoreceptors. An alternative view is that these changes are derived from increases in functional affinity, which result from multivalent presentation. While this mechanism is possible, evidence linking changes in ligand affinities with chemotactic activity is lacking. Equilibrium-binding constants for various ligands often do not correlate with ligand activities in bacterial chemotaxis assays [34, 35, 50, 51]. In contrast, a number of studies have implicated receptor localization in chemotaxis [38-41, 46, 52]. It has been shown, for example, that assembled tetramers of the chemoreceptor Tar are more active in in vitro signaling than are individual receptors or dimers [53]. Together, the present data and these results suggest that the differences in chemotactic activities for monovalent 1 versus multivalent 3 and 4 are due to their abilities to control the valency of receptor clusters. Based on these result, a mechanism in which systematic increases in ligand valency lead to changes in chemotactic responses by incorporation of additional receptors into clusters (as illustrated in Figures 7A-D) is proposed.

By generating synthetic molecules using ROMP that differ only in ligand valency, as opposed to ligand density or spacing, it has been shown that the valency of a ligand influences its ability to organize chemoreceptors and its ability to elicit a chemotactic response from those receptors. The results demonstrate that multivalent ligands of distinct valency (distinct or defined number of functional moieties), such as those described herein, can be used to tune cellular responses through changes in receptor organization. Further, ligand valency can be used to tune chemotactic responses of diverse bacteria (both E. coli and Bacillus subtilis) indicating that the methods of this invention are generally applicable to diverse cell types. The ROMP-based synthetic route to multivalent arrays is general [54] and can be employed to generate a variety of multivalent ligands or arrays which carry a variety of types and numbers of chemical signals that bind to cell receptors (cell surface receptors, transmembrane receptors and cytoplasmic receptors) and which as a result, likely mediated by lateral receptor reorganization, elicit a biological response. Control of the type of signal covalently bonded to the multivalent ligand and control of the spacing and number of signals presented on the ligand can be used to tune the type and magnitude of the response elicited.

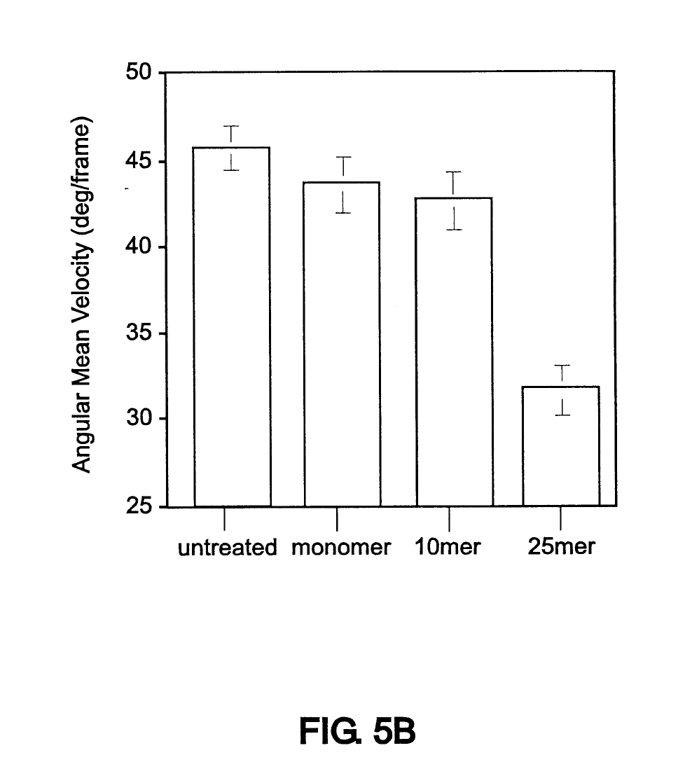

It has also been found that multivalent ligands that bind to one type of receptor can affect the biological response induced by binding of ligands to another type of receptor. Serine is another small molecule (in addition to galactose) which acts as a chemoattractant for bacteria, such as E. coli. Initial contact of E. coli cells with a multivalent ligand with galactose SREs, compounds 2 and 3 was followed, after a 2 min adaptation period, by addition of varying concentrations of serine. The chemoattractant effect of serine was enhanced about 30%, measured as average mean velocity (deg/frame) (see Figs. 5A and B), in the presence of multivalent ligands compared to serine in the absence of the multivalent ligand. It is believed that clustering of galactose-binding cell receptors by the multivalent ligand caused the enhancement of the response of the cell to the other chemoattractant serine, see Fig.1.

Modulation of Neutrophil ChemotaxisNeutrophil migration is an example of cell migration. Neutrophils migrate toward a number of different endogenous and exogenous substances. N-formyl peptides are one type of exogenous substance that is a chemoattractant for neutrophils [65], a bacterial transcription by product. Neutrophils have cell surface receptors which bind to the chemoattractant and can sense increasing concentration gradients of the chemoattractant. Neutrophils respond to the chemoattractant by migrating toward increased concentrations leading them to the site of infection, for example. In addition, and also in response to such chemoattractants neutrophils release intercellular signals that affect responses in other cells, particularly other immune systems cells. Multivalent ligands of this invention can be used to enhance the response of neutrophils to chemoattractants and enhance immune system clearance of infectious agents. Scheme 2 illustrates an exemplary N-formyl peptide 20 and an exemplary SRE for that N-formyl peptide 21 for use in multivalent ligands that modulate neutrophil migration. These signal groups (SREs) can be covalently or noncovalently bonded to ROMP scaffolds such as those illustrated in Scheme 2 (22 and 23). Scheme 3 provides exemplary linkers that can be employed in multivalent ligands carrying N- formyl-peptides.

Modulation of Immune ProcessesThe development of an immune response can be modulated via valency-dependent interactions of immune system cells with multivalent ligands of this invention. The recognition of foreign (non-self ) epitopes, cells, viruses or viral particles for clearance by the immune system is due in part to cell receptors that recognize the epitopes, cells, viruses or viral particle as foreign. In order for clearance to occur, the foreign signal must be recognized and there must be a B cell or T cell response to the foreign signal. Proper immune responses require activation and subsequent deactivation of B cells and T cells. Receptor clustering on B cells and T cells has been implicated in the production of an immune response.

Multivalent ligands of this invention which have one or more RE or SRE through which the ligand can bind to a B cell, T cell or other immune cell and which carry one or more antigens, epitopes can be employed to modulate the response of the immune cell (enhancing or decreasing immunogenicity of the antigen or epitope ). When the epitope or antigen is recognized as foreign (non-self) by the immune cell, cells or immune system in which an immune cell is found, then the multivalent ligand can be used to tolerize the immune cell, cells or immune system to the epitope or antigen. In this case, the epitope or antigen is that of a beneficial or clinical species (cell, particle, nucleic acid) or of a self cell (or tissue) that is incorrectly recognized as foreign (non-self). In contrast, a multivalent ligand of this invention can be used to sensitize or increase the sensitivity of the immune cell, cells or immune system to the foreign epitope or antigen enhance its immunogenicity and enhance the immune response to it. This method would be employed with a foreign epitope or antigen that was not beneficial, e.g., one associated with a pathogen. When the epitope or antigen is recognized as self by the immune cell, cells or immune system in which an immune cell is found, then the multivalent ligand can be used to sensitize the immune cell, cells or immune system to the self epitope or antigen. In this case, the epitope or antigen may be of a non-beneficial self cell or macromolecule, e.g., a cancer cell, or may be a foreign epitope or antigen that is incorrectly recognized as self. In contrast, a multivalent ligand of this invention can be used to tolerize the immune cell, cells or immune system to a self epitope or antigen that is incorrectly recognized as foreign. Methods for tolerization and sensitization are specifically exemplified hereafter.

The C3d complement fragment binds the CR2 receptor (CD21/CD19 complex) on B cells. The expression fusion product of the fusion of the cloned C3d gene fragment and the C-terminal region of hen egg lysozyme gene was able to increase immunogenicity significantly more (1000-fold) than the level achieved with the lysozyme combined with a strong adjuvant [62]. Scheme 4 illustrates an exemplary multivalent ligand containing two different signal groups 30 prepared from the ROMP polymer 29 by selective covalent bonding of the different signals. One of the signals is a hen egg lysozyme (HEL) peptide (specific for the A20 cell line): 103-117 NGMNAWVAWRNRCKG (SEQ ID NO: 1)[63] and the other is a 16-mer C3d peptide involved in binding to CR2: KNRWEDPGKQLYNVEA (SEQ ID NO: 2)[62]. This HEL peptide can be attached to the polymer backbone at the N-terminal amine (40) of the peptide or at a side group of a lysine near the end of the peptide (41 ):40:*GDGNGMNAWVAWRNR-CONH₂ (SEQ ID NO: 3) or41:DGNGMNAWVAWRNRGK*-CONH₂ (SEQ ID NO: 4)where * indicates the site of attachment. The C3d peptide can be attached to the multivalent ligand via the thiol of cysteine positioned at either end of the peptide(42 and 43):42:*CKNRWEDPGKQLYNVEA (SEQ ID NO: 5) or43:KNRWEDPGKQLYNVEAC* (SEQ ID NO: 6)Multivalent ligands containing signals 41 alone or in combination with 42 or 43 or 40 alone or in combination with 42 or 43 can induce an enhanced immune response compared to HEL its self. A multivalent ligand containing a plurality of peptide elements that are ligands for the CR2 receptor can cluster the CR2 receptor on the surface of the B cell and as demonstrated in the chemotaxis experiments can enhance the response of that B cell to other ligands, e.g., antigens. Multivalent ligands containing one or more bound CR2 ligands in combination with one or more bound antigens can cluster the CR2 receptor with the receptor that recognizes the antigen and thereby enhance the response of the B cell to the antigen. Clustering of CR2 with a receptor that recognized HEL(for example) on the B cell surface can enhance the response of the B cell for the HEL antigen and can result in an enhancement of immune response toward the HEL epitope. An alternative hen egg lysozyme peptide that can be employed in construction of multivalent ligands of this type is:44 :ELAAAMKRHGLDNYRGYSLGNWVCA (SEQ ID NO: 7).

CD22 is a B cell surface glycoprotein involved in cell adhesion and activation [64]. CD22 is important in the negative regulation of B cell antigen receptor signaling [74]. The structure recognized by CD22 is Sial2∀6 Gal∃14GlcNAc∃ (Scheme 5, compound 50). This signal can be attached to a ROMP polymer backbone as illustrated in Scheme 5 via a primary thiol group (compound 51). Multivalent ligands containing one or more ligands for CD22 (such as 51) in combination with one or more HEL epitopes (such as 42 or 43) attenuates the immune response to the HEL epitope.

Crosslinking (Aggregation) of Cells.

Many proteins, such as lectins and antibodies, possess multiple ligand binding sites. When these proteins bind to ligands immobilized on adjacent cell surfaces, the cells aggregate. Cell aggregation can be monitored easily, and this property has found use in the development of diagnostics for pathogen detection [75], therapeutics [76-78], blood typing tests [79], and other biotechnological applications [80-82]. Many lectins have been shown to have mitogenic activities that are dependent on the valency of the lectin. These mitogenic lectins, including ConcanavalinA (ConA), are thought to cluster glycoproteins on the surface of the target cell, activating mitogenic signals and inducing cell proliferation [67, 68]. Lectins have been useful tools for exploring signal transduction [69, 70] and cell growth [71, 72], and studies using them have elucidated possible functional roles for mammalian lectins, such as the galectins and selectins.

The effectiveness of multivalent proteins at instigating cell aggregation is determined by how tightly the protein binds to cell surface ligands. One effective way to increase the avidity of these interactions is to increase the number of ligand binding sites [83-85]. Research efforts have focused on favoring oligomer formation for lectins [86-87] or generating novel multimers of antibody scFv fragments [88]. Methods which further enhance the number of binding sites or favor the optimized orientation of these binding sites would increase the utility of these materials in many applications.

Lectins are a large class of saccharide-binding proteins, many of which are homo-oligomers assembled from two to four copies of identical subunits [89]. Lectins aggregate cells when they crosslink glycoproteins or glycolipids on adjacent cell surfaces. Aggregation can be modulated by altering the number of active monomers within the lectin oligomer. For example, the ability of the tetravalent mannose-binding plant lectin concanavalin A (Con A) to aggregate red blood cells is greatly decreased when the lectin is forced into a lower valency dimeric form by succinylation [87]. Increasing the valency of lectins may have the opposite effect, i.e. to enhance cell aggregation; however, methods have not been readily available for generating lectin complexes with higher order valencies. Because the valency of ROMP-derived materials can be altered systematically, the effect that the number of saccharide groups, such as mannose, bound to the ligand has on the number of lectins, such as ConA, assembled on a given scaffold can be investigated.

The precipitation of Con A depends on the clustering of Con A tetramers and this technique can be used to determine the stoichiometry of insoluble Con A - ligand complexes [90]. To investigate the formation of Con A clusters with multivalent ligands of this invention, ROMP-based scaffolds containing defined numbers of mannose residues, the monomer 9 and polymers 10-13 having n of 10, 25, 50, or 100, respectively, illustrated in Scheme 1 were prepared using ROMP methods [54]. Compounds 9-13 were contacted with Con A, monomeric compound 9 was unable to induce precipitation, but multivalent compounds 10-13, caused concentration-dependent precipitation of Con A. Precipitation results further indicated that the stoichiometry of ConA complexed with 10 (the 10-mer)is about 2:1 and that of complexes of ConA with 11 and 12 is approximately 4:1.

In contrast, dimeric succinylated Con A precipitated only with the highest valency multivalent ligand compounds tested, e.g., compound 12, and the complexes formed had a 4:1 (receptor:scaffold) stoichiometry in the precipitate. Thus, the number of mannose residues displayed by the scaffold is important in the formation of protein-scaffold complexes. Precipitation results were substantially confirmed with a transmission electron microscopy (TEM) technique in which clusters of biotinylated ConA with compounds 10-12 were labeled with a high density streptavidin gold particle. Compound 10 was observed to form dimers exclusively, while 11 was able to form both dimers and trimers and compound 12 formed both dimers and trimers as well, but favored trimeric clusters more than the other scaffolds.

The assembly of Con A clusters in solution can be monitored by fluorescence resonance energy transfer (FRET), in which fluorescein and tetramethylrhodamine (TMR) serve as donor and acceptor fluorophores [91, 92]. When these fluorophores are within approximately 80 Åthe fluorescein signal is quenched, such that fluorescein fluorescence should decrease when labeled Con A is assembled into clusters [93]. Compounds 9-12 were added to a solution of fluorescein- and TMR- labeled Con A. The fluorescence emission maximum of fluorescein was monitored to ascertain which scaffolds promoted the formation of Con A clusters. In agreement with the previous experiments, Con A clusters formed in the presence of multivalent ligands 10-12 but not with monomeric compound 9. The fluorescence quenching was dependent not only on scaffold valency, but also on ligand concentration. Quenching first increased as scaffold concentration increased and then decreased again as the concentration was increased further. The absence of quenching at high scaffold (multivalent ligand) concentrations indicates that Con A clusters are disfavored at these concentrations, likely because of site saturation. The high concentration of scaffold compared to Con A favors occupation of each ligand binding site on Con A by individual polymers precluding clustering of multiple lectins.

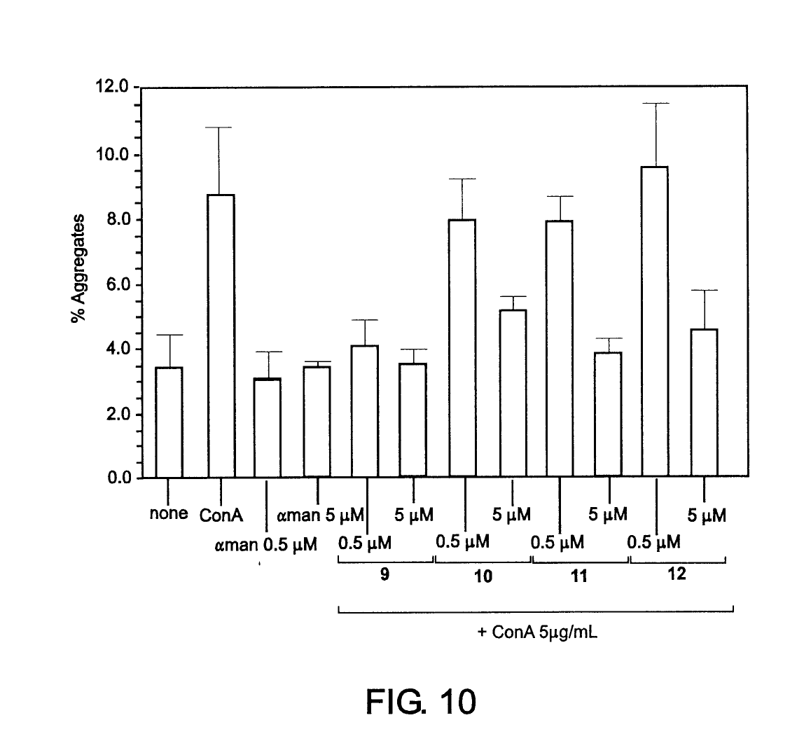

The ability of Con A clusters formed on ROMP-derived polymers to aggregate Jurkat cells was examined initially by light microscopy (see Fig. 10). Con A alone was able to induce some Jurkat cell aggregation even at low concentrations (5 µg/mL). When monovalent Con A ligands such as methyl α-D-mannopyranoside or 9 were premixed with Con A they inhibited aggregation, presumably by destabilizing Con A - cell interactions. For Jurkat cells, inhibition occurred even at low concentrations (0.5 µM) of monovalent ligands. Interestingly, multivalent compounds 10-12 did not inhibit Jurkat cell aggregation at 0.5 µM, a concentration shown to be optimum for Con A cluster formation under similar conditions. Increasing the concentration of the multivalent ligand 10-fold (5 µM) abolished aggregation activity, consistent with site saturation. Thus it is possible to alternatively inhibit or promote cell surface-lectin interactions by varying scaffold valency and multivalent ligand concentration. The ability of Con A complexed to multivalent ligands to interact with cell surfaces was thus tunable.

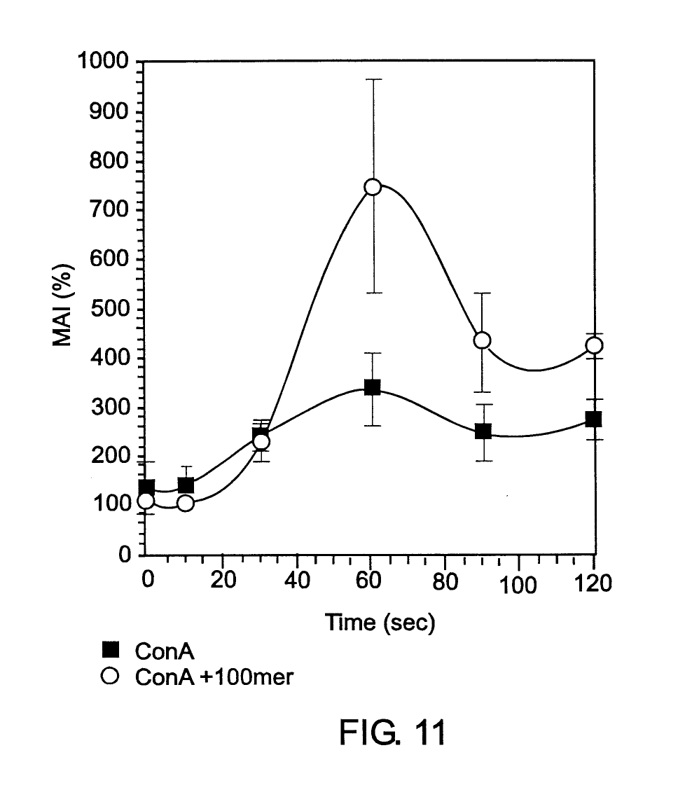

Further experiments were conducted which demonstrated that ConA-mediated agglutination of erythrocytes could be controlled by addition of multivalent ligands (compounds 9-13). Certain combinations of ConA and multivalent ligands exhibited enhanced agglutination of these cells compared to ConA itself, as shown in Fig. 11. In particular, a combination of ConA tetramer and multivalent ligand (compound 13) at concentration ratio 10:1 (based on tetrameric ConA and based on the number of mannose residues) exhibited significantly enhanced agglutination compared to ConA alone.

Complexes containing multiple Con A tetramers were assembled readily on compounds 10-13 when intermediate multivalent ligand concentrations were used, but were not detectable when the concentration of the scaffold was either too low or too high. The concentration range over which such complexes are formed depends upon the relative concentrations of ConA and multivalent ligand (based on the number of ligands, RE or SRE) and upon the valency of multivalent ligand. This is generally true for any complex of a multivalent ligand with any protein. The concentration range over which complexes of a multivalent ligand with one or more ConA (or such complexes with any lectin or more generally with any protein) can be readily determined for a particular application under particular conditions by assessing retention of function by ConA (or more generally the protein or lectin). Complexes of multivalent ligands with ConA will generally be formed, dependent upon the valency of the multivalent ligand and the particular experimental conditions, when the concentration range of the ligand (based on numbers of SRE, e.g., mannose) ranges from about 1:1 to over 100:1.

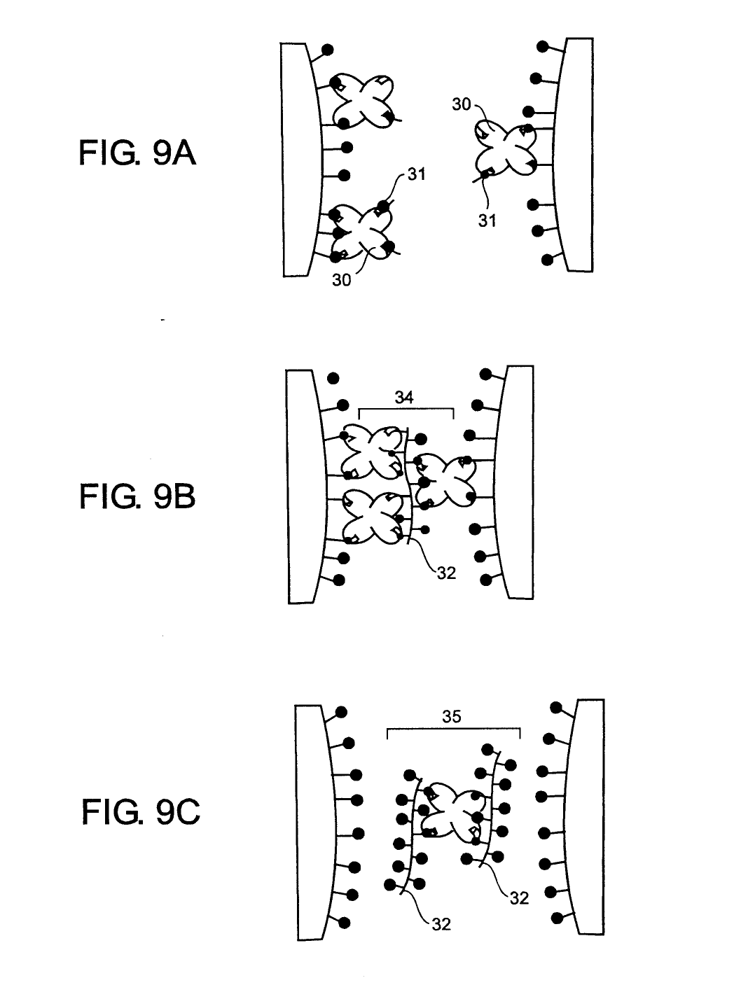

The result herein indicate generally that the valency and concentration of a multivalent ligand can be varied to control the assembly of lectin on to the multivalent scaffolds of these multivalent ligands. More specifically, the valency and concentration of ROMP-derived materials can be varied to control the formation of Con A clusters, as illustrated in Figs. 9A-C. Monovalent ligands (as well as low concentrations of multivalent ligands) ligands bind to lectin, but do not inhibit cell aggregation (Fig. 9A). Under conditions that favor lectinscaffold complexation, i.e., intermediate concentration levels of multivalent ligands, a plurality of lectins can be assembled on the multivalent ligand and the lectins retain free saccharide binding sites capable of interacting with cell surfaces (Fig. 9B). When multivalent ligand concentration is increased, lectin binding sites are saturated by binding to a plurality of multivalent ligands, lectin assembly is disfavored and lectins are not capable of interacting with cell surfaces (Fig. 9C). Thus, as illustrated, scaffold valency and ligand concentration can be controlled to assemble lectin clusters with multivalent ligands wherein the lectin retains cell binding activity. Further, scaffold valency and more importantly multivalent ligand concentration can be controlled to inhibit the cell aggregation function of lectins.

These results demonstrate that proteins, such as lectins, can be assembled on a polymeric scaffold, such as those provided by the multivalent ligands of this invention, and that the assembled proteins, including lectins, will retain biological function. Methods described herein can be employed to generate polymeric assemblies of one or more lectins, as well as polymeric assemblies of one or more antibodies or antibodies fragments, which retain the ability to bind to ligands (e.g., saccharides or epitopes). Methods herein are generally applicable to generation of assemblies of various chemical and biological species, particularly macromolecular species, including proteins, carbohydrates, nucleic acids though binding to recognition elements and signal recognition elements in a multivalent ligand.

Enhancement of Cell Toxicity Using Multivalent LigandsLectins, such as Con A, as well as agglutinins and phytohemaglutinins in general, can exhibit toxic effects in certain kinds of cells. Multivalent ligands carrying saccharide groups can complex with lectins, such as Con A, as discussed above. Complexes containing several lectin molecules complexed to an appropriately substituted multivalent ligand can function to aggregate cells, if binding sites on the lectin are not saturated by binding to the ligand groups. When higher multivalent ligand concentrations (dependent upon the specific conditions and applications, and dependent upon ligand valency) are employed, lectin binding sites can become saturated and cell aggregation by the lectin is then inhibited. Saturation of a given lectin by a given multivalent ligand can be readily determined empirically. Further, saturation of the function of any protein by a given multivalent ligand can be determined by assessing function of the complexed protein.

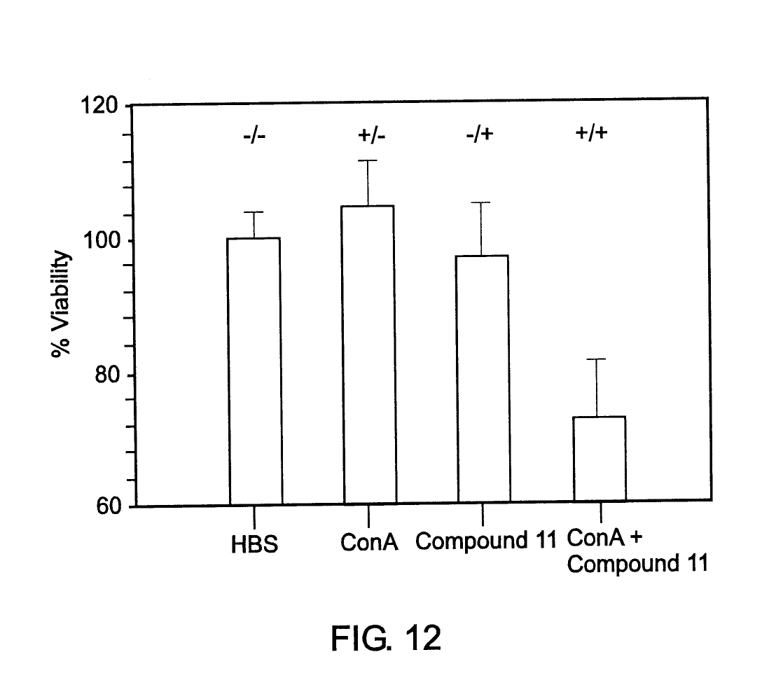

Complexes of a lectin with multivalent ligands have been found to exhibit cell toxicity that is enhanced over that of the lectin itself. As illustrated in Fig. 12, PC12 cells treated with 0.1 µM Con A (for 48 hr) exhibited no apparent loss of viability. In contrast, PC12 cells treated with combination of 0.1 µM Con A and 4 µM of compound 11 under the same conditions exhibit almost a 30% loss in viability. These results indicate that complexes of lectin with multivalent ligands of this invention in which the ratio of the concentrations of ligand to lectin is sufficiently high to saturate ligand binding sites of the lectin can trigger apoptosis in cells.

Generation of Multivalent Polymers ROMP was used to convert 1 to the series of oligomers 2-4 as previously described [55]. Similar conditions were employed in the synthesis of oligomers 6 and 7 [54]. Fluorescent polymer 8 was generated by specific end-labeling with a bifunctional capping agent [Scheme 7] and subsequent conjugation to the fluorophore BODIPY-TR (commercially available from Molecular Probes) [56]. Compounds 9-12 were the samples prepared and tested in reference [54]. The degree of polymerization (dp) for each compound was determined by ¹H NMR. Valency (n) is an approximation of the degree of polymerization (DP), where DP is the ratio of monomer to catalyst used in ROMP.

Video MicroscopyE. coli AW405, which exhibits wild-type chemotactic responses, from an overnight culture were grown in LB (Luria Bertani broth) to OD₅₅₀ of 0.4-0.6 and then washed twice with attractant-free chemotaxis buffer (10 mM potassium phosphate buffer, pH 7.0, 10 ΦM EDTA). Partially permeabilized bacteria (25 µM EDTA for 3 min. at room temperature, then quench with 50 µM CaCl₂) at an OD₅₅₀ of 0.1 were placed under a cover slip supported by additional cover slips in the method of Sager et al. [44]. (Permeabilization had no effect on bacterial chemotaxis toward galactose or 1 but was necessary for chemotaxis toward 4 [57]). Bacteria were allowed to adjust to contact with glass surface for 1-2 min. Attractant was added to achieve the final concentration indicated at a 5 ΦL final volume. The bacterial motion at 28 ΕC was recorded, and the paths were analyzed using the ExpertVision system. Paths derived from the first 5 to 15 seconds following the introduction of attractant were analyzed. Angular mean velocities varied approximately 14% between experiments performed on different days. Data were analyzed using the Q and Students tests.

Capillary Accumulation Assay E. coli from an overnight culture were grown in LB to OD₅₅₀ 0.4-0.6, washed twice with E. coli chemotaxis buffer, and then partially permeabilized. Bacteria were resuspended in chemotaxis buffer to an OD₅₅₀ 0.1 and utilized in the capillary accumulation assay at 30 ΕC for 60 min, as previously described [45]. B. subtilis OI1085 was grown from an overnight culture in T broth (1% tryptone, 0.2 mM MgCl₂, 0.5% NaCl, 0.01 mM MnCl₂) supplemented with 10 mM glucose and 0.5% glycerol, washed with B. subtilis chemotaxis buffer (10 mM phosphate buffer, pH 7.0, 10 ΦM EDTA, 0.5% glycerol, 0.3 mM (NH₄)₂SO₄), and capillary assays were performed at a final OD₅₅₀ 0.01 at 37 ΕC for 30 min [47]. The number of B. subtilis accumulated was normalized to 500 bacteria accumulated towards buffer alone. Results of capillary assays can be influenced by factors other than the activity of the attractant, such as metabolism of the substrate or toxicity [45, 58]. To exclude this possibility, we tested the ability of E. coli to utilize 1 as a sole carbon source. These experiments revealed that 1-4 are not toxic and that monomer 1 is not metabolized (data not shown). Data was analyzed using the Q and Students tests.

Immunofluorescence MicroscopyE. coli AW405 or RP1078 (cheW) were pretreated with buffer alone or with compounds 1, 3, 4, or 8 at 5 mM in a 10 ΦL total volume of chemotaxis buffer. After a 10 minute incubation at 30 ΕC, the bacteria were fixed (2% paraformaldehyde (PFA) in HEPES pH 7.0, 30 min., 4 ΕC), placed on poly-L-lysine treated cover slips in the bottom of 6-well plates, permeabilized with methanol, and labeled with anti-Tsr antibody (1:250) and fluorescein-labeled goat-anti-rabbit antibody (1:500) according to the procedure of Maddock and Shapiro [49]. Anti-Tsr antibodies recognize the conserved chemoreceptor cytoplasmic domain and are thus cross-reactive with multiple chemoreceptors. Some binding exclusion (exclusive 530 nm or 590 nm fluorescence at a pole) was seen in dual labeling experiments in which both antibody and 8 were used. Fluorescence microscopy was performed on a Zeiss Axioscope at 1000x using an oil immersion lens. Images were captured using IPLab Spectra 3.2 and Adobe PhotoShop 5.0.

Quantitative Precipitation Quantitative precipitations and analysis were carried out by a method modified from that previously described by Khan, et al [90]. Briefly, Con A (Vector Laboratories, Burlingame, CA) and scaffold were dissolved in precipitation buffer (0.1 M Tris-HCl pH 7.5, 90 µM NaCl, 1 mM CaCl₂, 1 mM MnCl₂), vortexed briefly to mix, and then incubated for 5 hours at room temperature (or 2 days at 4 °C for succinylated Con A). The final concentration of Con A tetramers was 30 µM (assuming Con A tetramers with a molecular weight of 104,000) and succinylated Con A dimers was 44 µM (assuming dimers with a mass of 52,000). White precipitates were pelleted by centrifugation at 5000 xg for 2 minutes. Supernatants were removed by pipet and pellets were gently washed twice with cold buffer. Pellets were then resuspended in 600 µL 100 mM methyl α-D-mannopyranoside (100 µL for succinylated Con A), and were completely dissolved after a 10 minute incubation at room temperature. Protein content was determined by measuring the absorbance at 280 nm by UV-vis spectroscopy on a Varian Cary 50 Bio using a 100 µL volume quartz cuvette. Measurements are the average of three independent experiments.

Transmission Electron Microscopy TEM methods were preformed essentially as previously described [96]. Con A tetramers were labeled with biotin using conditions that favored attachment of 1-2 copies of biotin residues. Biotinylated ConA tetramers were mixed with ligands of interest in solution and then contacted with an excess of streptavidin-conjugated 10 nm gold particles. Samples can be treated with 2% phosphotungstic acid (pH 7.0, 30 sec) to enhance contrast. Images of random fields were acquired for each treatment and analyzed for formation of ConA complexes. Gold particles within 25 nm of less of each other were considered to be part of a complex. This distance was based on the modeled length of the synthetic multivalent ligands used [23] and the structure of tetrameric ConA determined by X-ray crystallographic analysis[97].

Specifically, biotinylated Con A (2.3 µM) and scaffold (0.75 µM, mannose concentration) in PBS pH 7.2 were incubated for 15 minutes at room temperature before streptavidin - 10 nm gold (Sigma, St. Louis, MO) was added to a final concentration of 3.0 µM. Complexes were incubated at room temperature for 15 minutes and then placed onto carbon-coated Formvar-treated grids. Grids were air dried and viewed on a LEO Omega 912 Energy Filtering Electron Microscope (EFTEM). Images were at 12,500 x magnification, collected on a ProScan Slow Scan CCD camera, and analyzed in Adobe PhotoShop 5.0. Fields averaged between 5 and 50 gold particles and 15-20 fields were collected on each day for each treatment. Results are the average of results obtained on three separate experiments performed on independent samples on three separate days. Total number of gold particles collected on each day varied from about 80 to over 400.

Fluorescence Resonance Energy Transfer Fluorescein-Con A (Vector Laboratories, Burlingame, CA) and TMR-Con A (Sigma, St. Louis, MO) in phosphate buffered saline (PBS) pH 7.2 were mixed to afford final concentrations of 4 µg/mL and 0.4 µg/mL respectively. Scaffold was added in PBS to the final concentrations indicated, with a final volume of 200 µL. This solution was vortexed briefly and then incubated at room temperature for at least 15 minutes. No precipitates were observed in any of these samples. Fluorescence was determined on a Hitachi F-4500 fluorospectrophotometer using a 200 µL volume quartz cuvette, an excitation wavelength of 492 nm, emission wavelength of 515 nm, and 10 nm slit widths. Emission intensities are the average of 3-5 independent experiments with 3 scans performed during each experiment. Compounds 9-12 had negligible fluorescence at 515 nm. Curves were fit using the equation:%F = (%Fₘₐₓ x L)/(L + IC₅₀)where %F is the change in fluorescence relative to untreated, %Fₘₐₓ is the maximal recovery of fluorescence, L is the micromolar mannose concentration of scaffold, and IC₅₀ is the half-maximal concentration for inhibition of clustering.

Jurkat Cell Aggregation Jurkat cells were cultured and maintained as previously described [94]. Cells were washed three times in cold PBS and then treated with Hoescht 33342 (100 µg/mL) for 30 minutes at 30 °C. Cells were washed twice with cold PBS and then fixed for 30 minutes at 4 °C with 2% paraformaldehyde in HEPES pH 7.4. Fixed cells were washed twice and then treated in 200 µL final volume with premixed solutions of Con A and scaffold. A 2x solution of Con A and scaffold was prepared in PBS pH 7.2, vortexed briefly, and then incubated at 22 °C for 30 minutes before being added to cells. Cells, Con A solutions, and 100 µg/mL DNAse (to prevent cell aggregation by nucleic acid) were incubated at 22 °C for 30 minutes. Cells were pelleted at 400 xg, resuspended gently into 50 µL PBS, and then added to slides for visualization at 200 x magnification on a Zeiss Axioscope outfitted with the appropriate filter set. Approximately 100 - 200 cells were counted from random fields on each day. Clusters were scored for at least two cells in direct contact with each other and expressed as a percentage of the total number of objects (individual cells and clusters) counted. Results are summarized in Fig. 10. ROMP-derived ligands 9-12 alone were not able to cause cell aggregation. Images were captured in IPlab Spectra 3.2 and prepared in Adobe Photoshop 5.0.

Erythrocyte Agglutination ConA (53 nM, 5µg/mL) and ligand compound 13 (530 nM; per saccharide basis) were added in an end final volume of 100µL, PBS ph 7.2 in a 96-well plate. The complexes were incubated for 15 minutes at room temperature.

Cell Toxicity Experiments HBS buffer contained HEPES (10mM), NaCl (150mM), and CaCl₂ (1mM) at pH = 7.4. Concanavalin A (ConA) was obtained from Vector Labs (Burlington, CA) and was freshly diluted for all experiments. The concentration of the ConA stock solution was determined using

$${A}_{280}^{1 \%} = 13.7$$

(95). A single ConA dilution was then made and split for each sample. Ligands were then added from appropriate stock solutions at 5 times the desired final concentration. All samples had six replicates for each concentration. Control samples were used in each run that contained HBS alone, ConA in HBS and the highest concentration of ligand in HBS. Lysis controls were made by adding HBS buffer alone and adding lysis buffer after 48 h sample incubation.

Cell Culture: All cell culture reagents were obtained from GIBCO BRL unless otherwise noted. PC12 cells (ATCC: CRL-1721) were grown in media containing 84% (v/v) RPMI 1640 (with L-glutamine), 5% (v/v) heat inactivated fetal bovine serum, 10% (v/v) heat inactivated horse serum, and 1% penicillin/streptomycin (10000 units/ml), in a humidified incubator at 37°C and 5% CO₂. Low serum media contained 97.5%(v/v) RPMI 1640 (with L-glutamine), 0.5% inactivated fetal bovine serum, 1% (v/v) heat inactivated horse serum, and 1% penicillin /streptomycin (10000 units/ml). Cells were grown in T-flasks treated with collagen, and harvested at confluence by trypsinization (0.05% trypsin and 0.4 mM EDTA) followed by quenching with fresh medium. Cells were concentrated to pellet (2100 rpm for 10 min), aspirated then resuspended in fresh medium. The population was determined by haemocytometer and treatment with trypan blue, cells were then plated to 96-well plates (tissue culture treated obtained from CoStar, Corning NY) at ~15,000 cells/well. Plates were then incubated for 34 h to allow cells to adhere.

The medium was then removed and replaced with low serum medium(80 µL). Samples and controls in HBS (20 µL) were then added and incubated for 48 h at 37 C. After incubation 10 µL of a 5mg/mL solution of MTT (3-(4,5-dimethylthiazol-2-yl)-2,5-diphenyltetrazolium bromide (Sigma/Aldrich, Milwaukee, WI) in low serum RPMI medium was added to each well.

After 4 h, 100 µL of lysis buffer (50% dimethyl formamide/ 20% sodium dodecyl sulfate in HBS, pH = 4.7) was added and the cells were incubated overnight. The plate was then read on a plate reader (Biostar) at 570 nm. Percent cell viability was determined using the following equation:

$$\frac{{G}_{1} - {G}_{0}}{{G}_{con} {G}_{0}} = \% V$$

,where G₀ is the lysed cell control, G_{con} is control cells treated only with vehicle, and G₁ is a sample treated with peptide and vehicle. Results from an experiment in which cells are initially treated with multivalent polymer compound 11 followed by treatment with ConA and appropriate controls are illustrated in Fig. 11.

Those of ordinary skill in the art will appreciate in view of the descriptions herein that there are a variety of alternative structures, methods, procedure and techniques that can be readily applied or adapted to the practice of this invention other than those that have been specifically exemplified. It will be appreciated that there are a wide variety of designs for and methods for preparation of multivalent ligands with properties as described herein. It will also be appreciated that there are a wide variety of molecular scaffolds available for the productive presentation of SRE as well as a wide variety of SRE that can be applied or adapted to the methods described herein.

References:

1.Mammen, M., Choi, S.-K. & Whitesides, G. M. (1998). Polyvalent interactions in biological systems: implications for design and use of multivalent ligands and inhibitors. Angew. Chem., Int. Ed. Engl. 37, 2755-2794.

2. Kiessling, L. L. & Pohl, N. L. (1996). Strength in numbers: Non-natural polyvalent carbohydrate derivatives. Chem. Biol. 3, 71-77.

3.Williams, L. T. (1989). Signal transduction by the platelet-derived growth factor receptor. Science 243, 1564-1570.

4. Heldin, C.-H. (1995). Dimerization of cell surface receptors in signal transduction. Cell 80, 213-223.

5. Fire, E., Brown, C. M., Roth, M. G., Henis, Y. I. & Petersen, N. O. (1997). Partitioning of proteins into plasma membrane microdomains: clustering of mutant influenza virus hemagglutinins into coated pits depends on the strength of the internalization signal. J. Biol. Chem. 272, 29538-29545.

6. Germain, R. N. (1997). T-cell signaling: The importance of receptor clustering. Curr. Biol. 7, R640-644.

7. Holowka, D. & Baird, B. (1996). Antigen-mediated IgE receptor aggregation and signaling: A window on cell surface structure and dynamics. Annu. Rev. Biophys. Biomol. Struct. 25, 79-112.

8. Monks, C. R. F., Freiberg, B. A., Kupfer, H., Sciaky, N. & Kupfer, A. (1998). Three-dimensional segregation of supramolecular activation clusters in T-cells. Nature 395, 82-86.

9. Yap, A. S., Brieher, W. M., Pruschy, M. & Gumbiner, B. M. (1997). Lateral clustering of the adhesive ectodomain: a fundamental determinant of cadherin function. Curr. Biol. 7, 308-315.

10. Hato, T., Pampori, N. & Shattil, S. J. (1998). Complementary roles for receptor clustering and conformational change in the adhesive and signaling functions of integrin ∀_{IIb}∃₃. J. Cell Biol. 141, 1685-1695.

11. Kuduk, S. D., Schwarz, J. B., Chen, X.-T., et al. (1998). Synthetic and immunological studies on clustered modes of mucin-related Tn and TF O-linked antigens: The preparation of a glycopeptide-based vaccine for clinical trials against prostate cancer. J. Am. Chem. Soc. 120, 12474-12485.

12. Dintzis, R. Z., Okajima, M., Middleton, M. H., Greene, G. & Dintzis, H. M. (1989). The immunogenicity of soluble haptenated polymers is determined by molecular mass and hapten valence. J. Immunol. 143, 1239-1244.

13. Schlessinger, J. (1988). Signal transduction by allosteric receptor oligomerization. TIBS 13, 443-447.

14. Klemm, J. D., Schreiber, S. L. & Crabtree, G. R. (1998). Dimerization as a regulatory mechanism in signal transduction. Annu. Rev. Immunol. 16, 569-592.

15. Weiss, A. & Schlessinger, J. (1998). Switching signals on or off by receptor dimerization. Cell 94, 277-280.

16. Metzger, H. (1992). Transmembrane signaling: The joy of aggregation. J. Immunol. 149, 1477-1487.

17. Spencer, D. M., Wandless, T. J., Schreiber, S. L. & Crabtree, G. R. (1993). Controlling signal transduction with synthetic ligands. Science 262, 1019-1024.

18. Tian, S.-S., Lamb, P., King, A. G., et al. (1998). A small, nonpeptidyl mimic of granulocyte-colony-stimulating factor. Science 281, 257-259.

19. Kramer, R. H. & Karpen, J. W. (1998). Spanning binding sites on allosteric proteins with polymer-linked ligand dimers. Nature 395, 710-713.

20. Lees, A., Morris, S. C., Thyphronitis, G., et al. (1990). Rapid stimulation of large specific antibody responses with conjugates of antigen and anti-IgD antibody. J. Immunol. 145, 3594-3600.

21. Sigal, G. B., Mammen, M., Dahmann, G. & Whitesides, G. M. (1996). Polyacrylamides bearing pendant ∀-sialoside groups strongly inhibit agglutination of erythrocytes by influenza virus: The strong inhibition reflects enhanced binding through cooperative polyvalent interactions. J. Am. Chem. Soc. 118, 3789-3800.

22. Kiessling, L. L. & Strong, L. E. (1998). Bioactive polymers. Top. Organomet. Chem. 1, 199-231.

23. Kanai, M., Mortell, K. H. & Kiessling, L. L. (1997). Varying the size of multivalent ligands: The dependence of concanavalin A binding on neoglycopolymer length. J. Am. Chem. Soc. 119, 9931-9932.

24. Lynn, D. M., Kanaoka, S. & Grubbs, R. H. (1996). Living ring-opening metathesis polymerization in aqueous media catalyzed by well-defined ruthenium carbene complexes. J. Am. Chem. Soc. 118, 784-790.

25. Arimoto, H., Nishimura, K., Kinumi, T., Hayakawa, I. & Uemura, D. (1999). Multivalent polymer of vancomycin: Enhanced antibacterial activity against VRE. Chem. Comm. 15, 1361-1362.

26. Gibson, V. C., Marshall, E. L., North, M., Robson, D. A. & Williams, P. J. (1997). Thymine functionalized polymers via living ring-opening metathesis polymerisation. J. Chem. Soc., Chem Commun. 1095-1096.

27. Sanders, W. J., Katsumoto, T. R., Bertozzi, C. R., Rosen, S. D. & Kiessling, L. L. (1996). L-selectin-carbohydrate interactions: Relevant modifications of the Lewis x trisaccharide. Biochemistry 35, 14862-14867.

28. Gordon, E. J., Sanders, W. J. & Kiessling, L. L. (1998). Synthetic ligands point to cell surface strategies. Nature 392, 30-31.

29.Parkinson, J. S. (1993). Signal transduction schemes of bacteria. Cell 73, 857-871.

30. Hazelbauer, G. L., Berg, H. C. & Matsumura, P. (1993). Bacterial motility and signal transduction. Cell 73, 15-22.

31. Alon, U., Surette, M. G., Barkai, N. & Leibler, S. (1998). Robustness in bacterial chemotaxis. Nature 393, 18-19.

32. Barkal, N. & Leibler, S. (1997). Robustness in simple biochemical networks. Nature 387, 913-917.

33. Grebe, T. W. & Stock, J. (1998). Bacterial chemotaxis: The five sensors of a bacterium. Curr. Biol. 8, R154-R157.

34. Adler, J., Hazelbauer, G. L. & Dahl, M. M. (1973). Chemotaxis towards sugars in Escherichia coli. J. Bacteriol. 115, 824-847.

35. Hazelbauer, G. L. & Adler, J. (1971). Role of the galactose binding protein in chemotaxis of Escherichia coli toward galactose. Nature New Biol. 230, 101-104.

36. Silversmith, R. E. & Bourret, R. B. (1999). Throwing the switch in bacterial chemotaxis. Trends Microbiol. 7, 16-22.

37. Mesibov, R., Ordal, G. W. & Adler, J. (1973). The range of attractant concentrations for bacterial chemotaxis and the threshold and size over this range. J. Gen. Physiol. 62, 203-223.

38. Jasuja, R., Keyoung, J., Reid, G. P., Trentham, D. R. & Khan, S. (1999). Chemotactic responses of Escherichia coli to small jumps of photoreleased L-aspartate. Biophys. J. 76, 1706-1719.

39. Bray, D., Levin, M. D. & Morton-Firth, C. J. (1998). Receptor clustering as a cellular mechanism to control sensitivity. Nature 393, 85-88.

40. Levit, M. N., Liu, Y. & Stock, J. B. (1998). Stimulus response coupling in bacterial chemotaxis: receptor dimers in signaling arrays. Mol. Microbiol. 30, 459-466.

41. Duke, T. A. J. & Bray, D. (1999). Heightened sensitivity of a lattice of membrane receptors. Proc. Natl. Acad. Sci. USA 96, 10104-10108.

42. Berg, H. C. & Brown, D. A. (1972). Chemotaxis in Escherichia coli analysed by three dimensional tracking. Nature 239, 500-504.

43. Amsler, C. D. (1996). Use of computer-assisted motion analysis for quantitative measurements of swimming behavior in petrichously flagellated bacteria. Anal. Biochem. 235, 20-25.

44. Sager, B. M., Sekelsky, J. J., Matsumura, P. & Adler, J. (1988). Use of a computer to assay motility in bacteria. Anal. Biochem. 173, 271-277.

45. Adler, J. (1973). A method for measuring chemotaxis and use of the method to determine optimum conditions for chemotaxis by Escherichia coli. J. Gen. Microbiol. 74, 77-91.

46. Barnakov, A. N., Downing, K. H. & Hazelbauer, G. L. (1994). Studies of the structural organization of a bacterial chemoreceptor by electron microscopy. J. Struct. Biol. 112, 117-124.

47. Ordal, G. W., Villani, D. P. & Rosendahl, M. S. (1979). Chemotaxis towards sugars by Bacillus subtilis. J. Gen. Microbiol. 115, 167-172.

48. Ordal, G. W. (1985). Bacterial chemotaxis: Biochemistry of behavior in a single cell. Crit. Rev. Microbiol. 12, 95-130.

49. Maddock, J. R. & Shapiro, L. (1993). Polar location of the chemoreceptor complex in the Escherichia coli cell. Science 259, 1717-1723.

50. Boos, W. (1969). The galactose binding protein and its relationship to the ∃-methylgalactoside permease from Escherichia coli. Eur. J. Biochem. 10, 66-73.

51. Yaghmai, R. & Hazelbauer, G. L. (1993). Strategies for differential sensory responses mediated through the same transmembrane receptor. EMBO J.12, 1897-1905.

52. Li, G. & Weis, R. M. (2000). Covalent modification regulates ligand binding to receptor complexes in the chemosensory system of Escherichia coli. Cell 100, 357-365.

53. Cochran, A. G. & Kim, P. S. (1996). Imitation of Escherichia coli aspartate receptor signaling in engineered dimers of the cytoplasmic domain. Science 271, 1113-1116.

54. Strong, L. E. & Kiessling, L. L. (1999). A general synthetic route to defined, biologically active multivalent arrays. J. Am. Chem. Soc. 121, 6193-6196.

55. Manning, D. D., Strong, L. E., Hu, X., Beck, P. J. & Kiessling, L. L. (1997). Neoglycopolymer inhibitors of the selectins. Tetrahedron 53, 11937-11952.

56. Gordon, E. J., Gestwicki, J. E., Strong, L. E. & Kiessling, L. L. (2000). A bifunctional capping agent for ROMP: synthesis and cell binding of a fluorescent neoglycopolymer. Chem. Biol. 7, 9-16.

57. Leive, L. & Kollin, V. (1967). Controlling EDTA treatment to produce permeable Escherichia coli with normal metabolic processes. Biochem. Biophys. Res. Comm. 28, 229-236.

58. Adler, J. (1966). Chemotaxis in bacteria. Science 153, 708-716.

59. Stoddard, B. L. & Koshland, D. E. J. (1992). Prediction of the structure of a receptor-protein complex using a binary docking method. Nature 358, 774-776.

60. Zhang, Y., Gardina, P. J., Kuebler, A. S., et al. (1999). Model of maltose-binding protein/chemoreceptor complex supports intrasubunit signaling mechanism. Proc. Natl. Acad. Sci. USA 96, 939-944.

61.Moudgil, K., Sekiguchi, D., Kim, S., & Sercarz, E. (1997)Immunodominance is independent of structural constraints, J. Immunology 159 (6), 2574-2579.

62.Lou, D., & Kohler, H. (1998) Enhanced molecular mimicry of CEA using photoaffinity crosslinked C3d peptide. Nature Biotechnology , 16, 458-462.

63.Gapin, L., et al. (1998) Antigen presentation by dendritic cells focuses T cell responses against immunodominant peptides: studies in the hen egg-white lysozyme (HEL) model, J. Immunology, 160, 1556-1564.

64.Powell, L., & Varki, A. (1994)The oligosaccharide binding specificities of CD22∃, a sialic acid-specific lectin of B cells, J. Biol. Chem., 269, 10628-10636.

65.Ye, R., & Boulay, F., (1997) Leukocyte Chemoattractant Receptors, Advances in Pharmacology 39:221.

66.PCT application WO 98/46270, (Advanced Medicine) published October 22, 1998.

67.Ramaschi, G., Torti, M., Sinigaglia, F. & Balduini, C. (1993). Intracellular Calcium Mobilization is Triggered by Clustering of Membrane Glycoproteins in Concanavalin A-stimulated Platelets. Cell Biochem. Function 11, 241-249.

68.Kaplan, M.R., Trubniykov, E. & Berke, G. (1997). Fluorescence Depolarization as an Early Measure of T Lymphocyte Stimulation. J. Immunol. Methods 201, 15-24.

69.Walzel, H., Blach, M., Hirabayashi, J., Kasai, K.-I. & brock, J. (2000). Involvement of CD2 and CD3 in Galectin-1 Induced Signaling in Human Jurkat T-cells. Glycobiology 10, 131-140.

70.Wenzel-Seifert, K., Krautwurst, D., Lentzen, H. & Siefert, R. (1996). Concanavalin A and Mistletoe Lectin I Differentially Activate Cation Entry and Exocytosis in Human Neutrophils: Lectins May Activate Multiple Subtypes of Cation Channels. J. Leukocyte Biol. 60, 345-355.

71.Stewart, R., Allan, D.W. & McCraig, C.D. (1996). Lectins Implicate Specific Carbohydrate Domains in Electric Field Stimulated Nerve Growth and Guidance. J. Neurobiol. 30, 425-437.

72.Rott, O., Charreire, J. & Cash, E. (1996). Influenza A Virus Hemagglutinin is a B-cell-superstimulatory Lectin. Med. Microbiol. Immunol. 184,185-193.

73.Singh, R.S., Tiwary, A.K. & Kennedy, J.F. (1999). Lectins: Sources, Activities, and Applications. Crit. Rev. Biotech. 19, 145-178.

74.Blasioli, J. Paust, S. & Thomas, M.L. (1999). Definition of the Sites of Interaction between the Protein Tyrosine Phosphatase SHP-1 and CD22. J. Biol. Chem. 274, 2303-2307.

75. Kemp, B. E., Rylatt, D. B., Bundesen, P. G., et al. (1988). Autologous red cell agglutination assay for HIV-1 antibodies: Simplified test with whole blood. Science 241, 1352-1354.

76. Kawauchi, H., Hosono, M., Takayanagi, Y. & Nitta, K. (1993). Agglutinins from aquatic insects-tumor cell agglutination activity. Experientia 49, 358-361.

77. Coloma, M. J. & Morrison, S. L. (1997). Design and production of novel tetravalent bispecific antibodies. Nature Biotechnol. 15, 159-163.

78. Segal, D. M., Weiner, G. J. & Weiner, L. M. (1999). Bispecific antibodies in cancer therapy. Curr. Opin. Immunol. 11, 558-562.

79. Furata, M., Uchikawa, M., Ueda, Y., et al. (1998). Construction of mono- and bivalent human single-chain Fv fragments against the D antigen in the Rh blood group: Multimerization effect on cell agglutination and application to blood typing. Protein Eng. 11, 233-241.

80. Singh, R. S., Tiwary, A. K. & Kennedy, J. F. (1999). Lectins: Sources, activities, and applications. Crit. Rev. Biotech. 19, 145-178.

81. Matsuya, Y. & Yamane, I. (1985). Cell hybridization and cell agglutination 1. Enhancement of cell hybridization by lectins. J. Cell Sci. 78, 263-271.

82. Takamatsu, H., Kawajiri, H., Takahashi, Y., Manaf Ali, A. & Yoshimoto, T. (1999). Continuous antibody production by phytohemagglutinin-L-aggregated hybridoma cells. J. Immunol. Methods 223, 165-170.

83. Crothers, D. M. & Metzger, H. (1972). The influence of polyvalency on the binding properties of antibodies. Immunochem. 9, 341-357.

84. Mammen, M., Choi, S.-K. & Whitesides, G. M. (1998). Polyvalent interactions in biological systems: implications for design and use of multivalent ligands and inhibitors. Angew. Chem. Int. Ed. Engl. 37, 2755-2794.

85. Kiessling, L. L. & Pohl, N. L. (1996). Strength in numbers: Non-natural polyvalent carbohydrate derivatives. Chem. Biol. 3, 71-77.

86. Torti, M., Ramaschi, G., Sinigaglia, F. & Balduini, C. (1995). Dual mechanism of protein-tyrosine phosphorylation in concanavalin A-stimulated platelets. J. Cell. Biochem. 57, 30-38.

87. Osawa, T. & Beppu, M. (1987). Cross-linked derivatives of concanavalin A. Methods Enzymol. 150, 17-28.

88. Hudson, P. J. & Kortt, A. A. (1999). High avidity scFv multimers; diabodies and triabodies. J. Immunol. Methods 231, 177-189.

89. Weis, W. I., Taylor, M. E. & Drickamer, K. (1998). The C-type lectin superfamily in the immune system. Immunol. Rev. 163, 19-34.

90. Khan, M. I., Mandal, D. K. & Brewer, C. F. (1991). Interactions of concanavalin A with glycoproteins. A quantitative precipitation study of concanavalin A with the soybean agglutinin. Carbohydr. Res. 213, 69-77.

91. Ballerstadt, R. & Schultz, J. S. (1997). Competitive-binding assay method based on fluorescence quenching of ligand held in close proximity by a multivalent receptor. Anal. Chim. Acta 345, 203-212.

92. Burke, S. D., Zhao, Q., Schuster, M. C. & Kiessling, L. L. (2000). Synergistic formation of soluble lectin clusters by a templated multivalent saccharide ligand. J. Am. Chem. Soc. 122, 4518-4519.

94. Gordon, E. J., Gestwicki, J. E., Strong, L. E. & Kiessling, L. L. (2000). Synthesis of end-labeled multivalent ligands for exploring cell-surface-receptor-ligand interactions. Chem. Biol. 7, 9-16.

95.Gupta D, Dam TK, Oscarson S, Brewer CF.(1997) Thermodynamics of lectin-carbohydrate interactions. Binding of the core trimannoside of asparagine-linked carbohydrates and deoxy analogs to concanavalin A. J Biol Chem. 272(10):6388-92.

96.Gestwicki, J.E., Strong, L. E. & Kiessling, L. L.(2000) Visualization of single multivalent receptor-ligand complexes by transmission electron microscopy. Angew. Chem. Int. Ed. 39, No. 24, 4567-4570.

97.Parkin, S., Rupp, B., Hope, H. (1996) Atomic resolution structure of concanavalin A. Acta Crystallogr. Sect. D 52:1161-1168.

98.Hunig, S., Muller, H.R., Their, W. Angew. Chem. Int. Ed. Engl. (1965) 4:271-382.

99.Hart, D.J.; Hong, W.-P.; Hsu, L-Y. J. Org. Chem. (1987) 52: 4665-4673.

100.Johnson, R.A., Sharpless, K.B. (1993) In Catalytic Asymmetric Synthesis, Ojima, I. (ed.)VCH: New York .

101.Walsh, P.J., Bennani, Y.L., Sharpless, K.B. (1993) Tetrahedron Lett. 34:5545-5548.

102.Kiss et al. (1997) Gut 40:253.

### [Schemes]

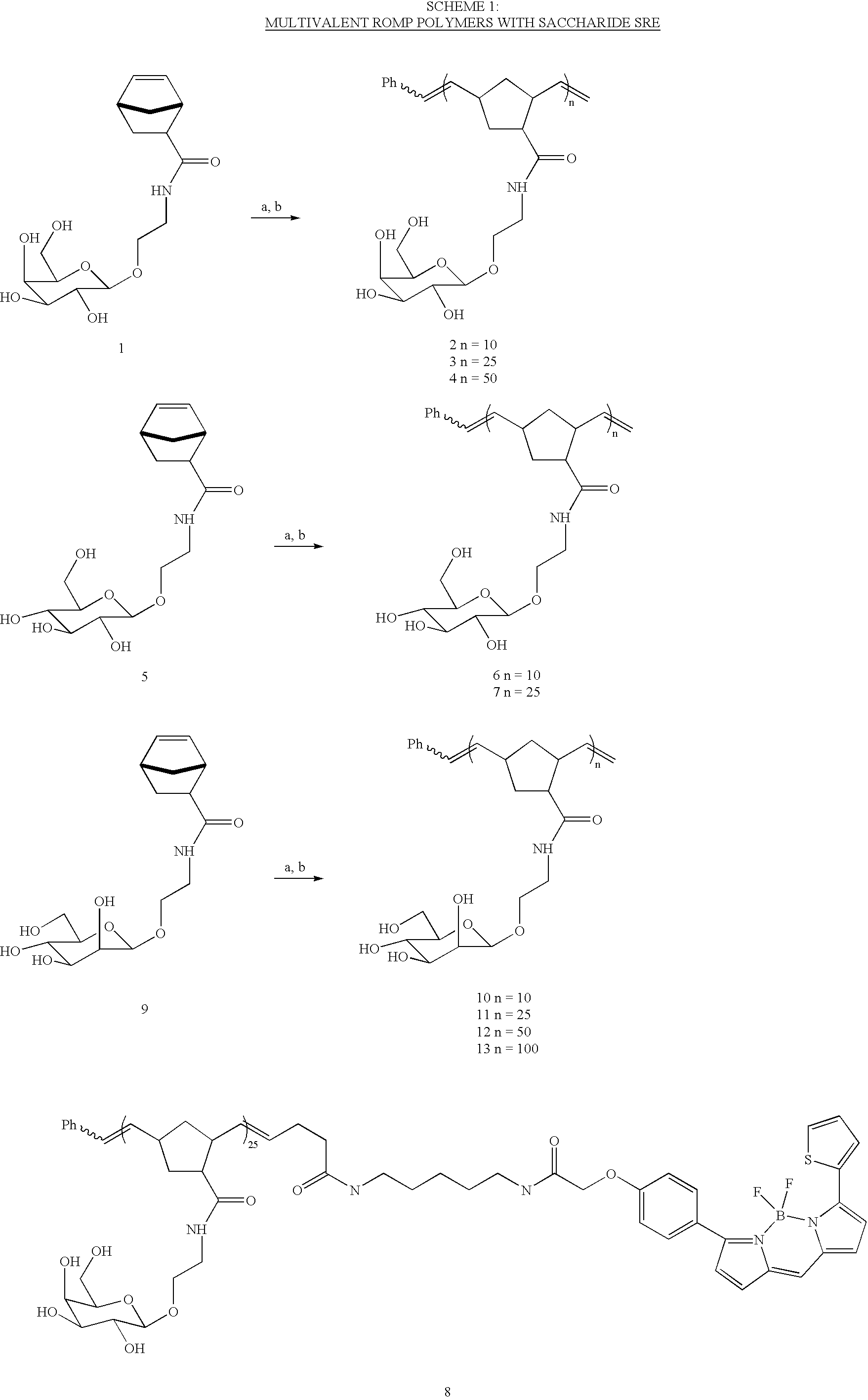

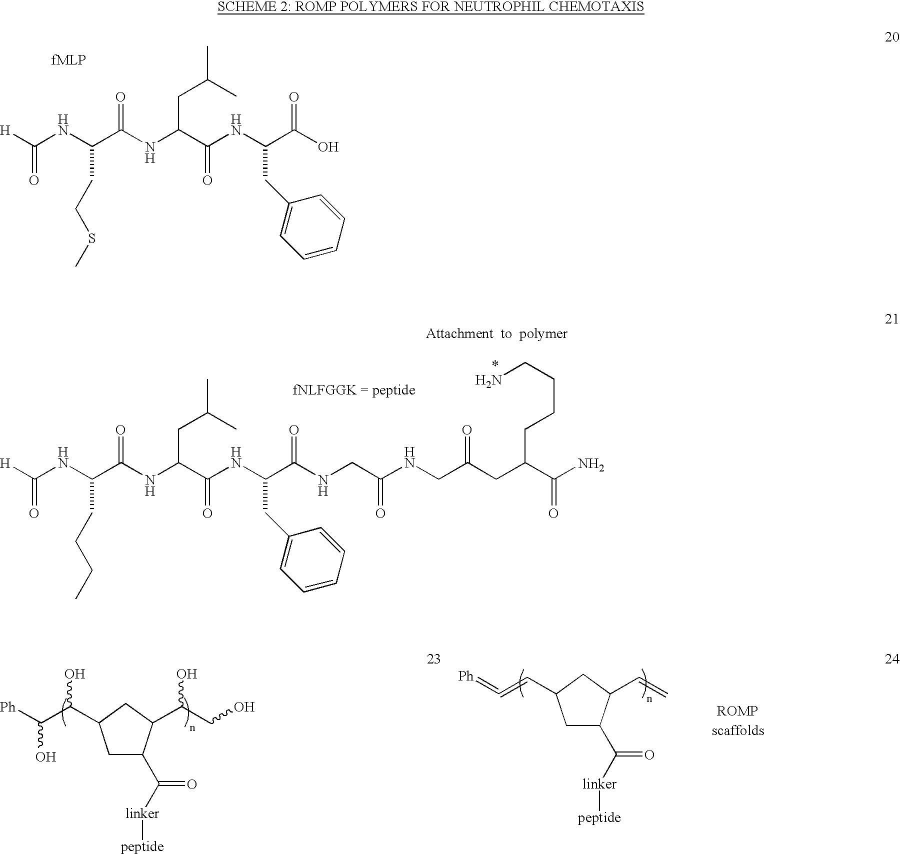

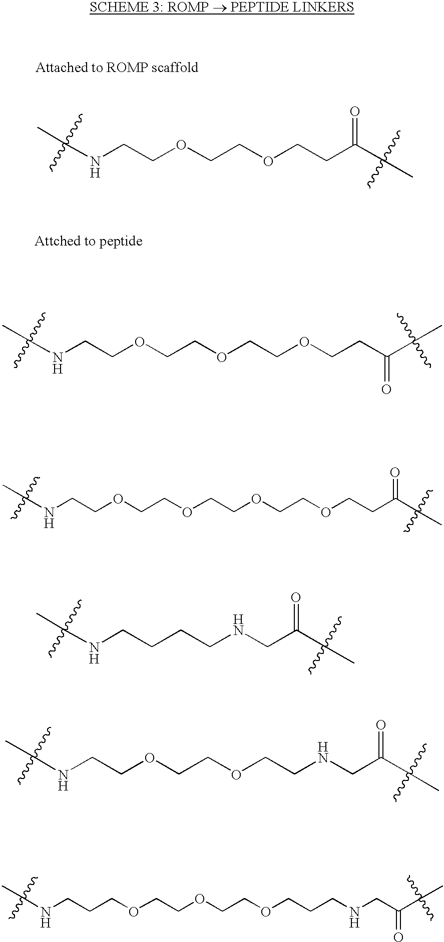

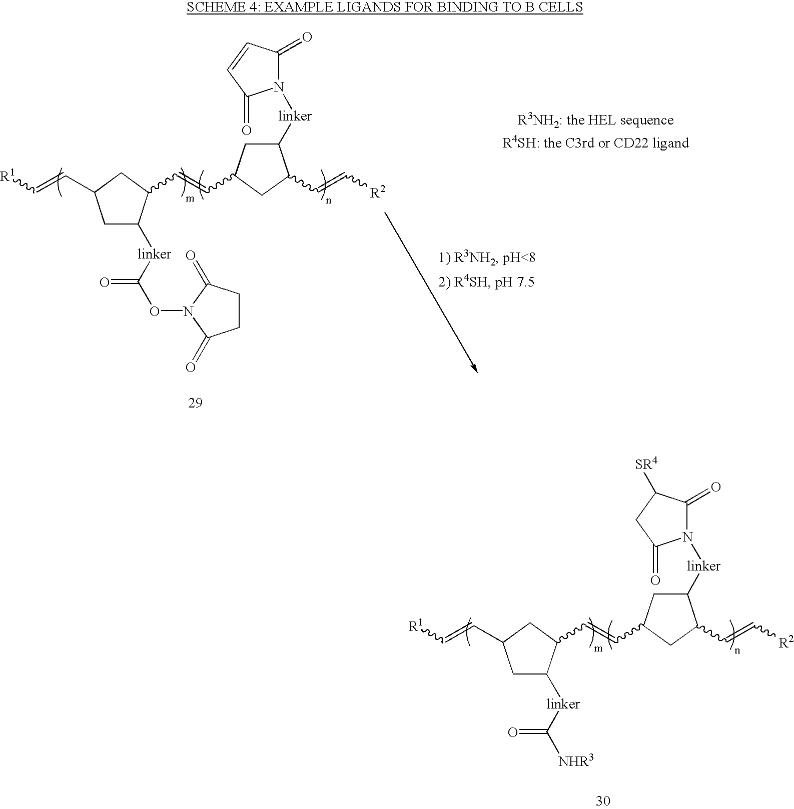

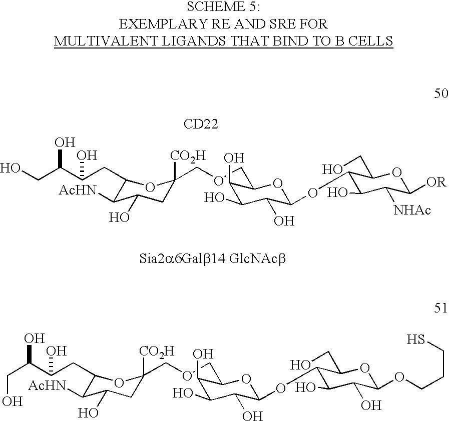

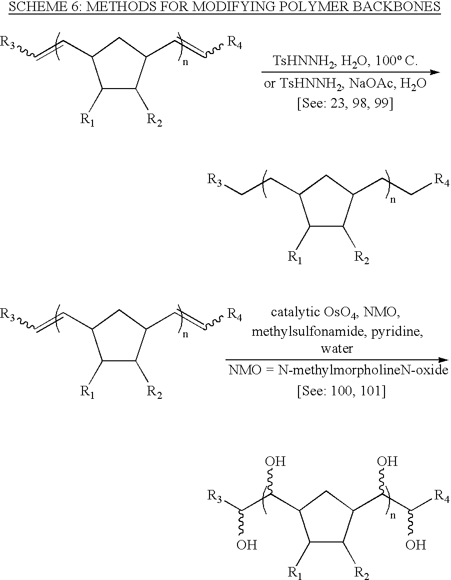

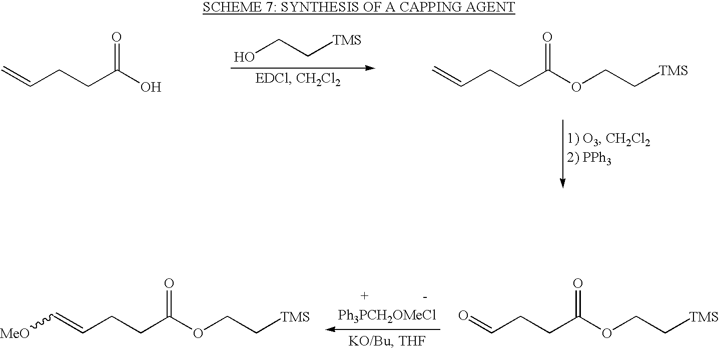

Claims

1. A method for inducing a biological response in a biological system comprising one or more receptors which comprises the step of introducing into the biological system a multivalent ligand which comprises a plurality of signal recognition elements recognized by at least one of the receptors and bonded to a molecular scaffold.

2. The method of claim 1 wherein the biological system comprises a cell having one or more cell receptors to which at least one of the signal recognition elements bind.

3. The method of claim 2 wherein binding of the signal recognition element to the receptor induces an intracellular response and/or an intercellular response.

4. The method of claim 1 wherein the cell is a prokaryotic cell.

5. The method of claim 4 wherein the multivalent ligand modulates signal transduction mediated by a two component system.

6. The method of claim 5 wherein the biological response is chemotaxis.

7. The method of claim 6 wherein the signal recognition element is a saccharide and the multivalent ligand comprises a plurality of the saccharides that function as chemoattractants covalently attached to a molecular scaffold.

8. The method of claim 7 wherein the saccharide is glucose or galactose.

9. The method of claim 6 wherein the multivalent ligand enhances chemotaxis of the prokaryotic cell.

10. The method of claim 6 wherein the multivalent ligand inhibits chemotaxis of the prokaryotic cell.

11. The method of claim 5 wherein the biological response is the formation of a biofilm.

12. The method of claim 11 wherein the multivalent ligand inhibits biofilm formation.

13. The method of claim 11 wherein the multivalent ligand enhances biofilm formation.

14. The method of claim 5 wherein the biological response is nutrient uptake.

15. The method of claim 14 wherein the multivalent ligand prevents or inhibits nutrient uptake.

16. The method of claim 14 wherein the multivalent ligand enhances nutrient uptake.

17. The method of claim 2 wherein the cell is a eukaryotic cell.

18. The method of claim 17 wherein the multivalent ligand modulates signal transduction mediated by G-protein coupled receptors.

19. The method of claim 18 wherein signal transduction is mediated by receptors.

20. The method of claim 17 wherein the eukaryotic cell is an epithelial cell or an endothelial cell.

21. The method of claim 17 wherein the eukaryotic cell is a cell of the immune system.

22. The method of claim 21 wherein the eukaryotic cell is a lymphocyte or an leukocyte.

23. The method of claim 21 wherein the eukaryotic cell is a neutrophil.

24. The method of claim 23 wherein the response is chemotaxis.

25. The method of claim 24 wherein one or more of the signal recognition elements is a formylated peptide and wherein the multivalent ligand comprises a plurality of formylated peptides covalently bonded to a molecular scaffold.

26. The method of claim 25 wherein the multivalent ligand inhibits neutrophil chemotaxis.

27. The method of claim 25 wherein the multivalent ligand enhances neutrophil chemotaxis.

28. The method of claim 17 wherein the biological response is the release of an intracellular signal by the cell.

29. The method of claim 28 wherein the multivalent ligand initiates or enhances the release of the intracellular signal.

30. The method of claim 21 wherein the cell is a B-cell or a T-cell.

31. The method of claim 30 wherein the multivalent ligand comprises a signal recognition element that is an epitope foreign to the organism from which the B-cell or T-cell originates.

32. The method of claim 31 wherein the multivalent ligand further comprises a signal recognition element that binds to a cell surface receptor of a B-cell or a T-cell.

33. The method of claim 32 wherein the multivalent ligand functions to enhance immunogenicity of the foreign epitope.

34. The method of claim 30 wherein the multivalent ligand comprises a signal recognition element that is an epitope recognized as a self epitope by the B- cell or T-cell.

35. The method of claim 34 wherein the multivalent ligand further comprises a signal recognition element that binds to a cell surface receptor of a B-cell or a T-cell.

36. The method of claim 35 wherein the multivalent ligand functions to sensitize the cell to the self epitope.

37. The method of claim 36 wherein the epitope is an epitope that is characteristic of a cancer cell.

38. The method of claim 30 wherein the multivalent ligand comprises at least one signal recognition element that is a self epitope which is recognized as a foreign epitope by the B-cell or T-cell.

39. The method of claim 38 wherein the multivalent ligand further comprises a signal recognition element that binds to a cell surface receptor of a B-cell or a T-cell.

40. The method of claim 39 wherein the multivalent ligand functions to tolerize the cell to the self epitope that is recognized as a foreign epitope by the B-Cell or T-cell.

41. The method of claim 1 wherein the multivalent ligand reorganizes receptors on the surface of a cell to modulate the biological response.

42. The method of claim 41 wherein the relative positions of different receptors on the cell surface is changed to modulate the response.

43. The method of claim 42 wherein interactions between cell surface receptors are changed to modulate the response.

44. The method of claim 1 wherein the biological response is an immune response to an antigen or epitope that is foreign to the biological system.

45. The method of claim 44 wherein the multivalent ligand modifies the immune response the foreign antigen or epitope and wherein the multivalent ligand comprises a signal recognition element that is an epitope or antigen that is recognized as foreign by the immune cell, cells or immune system that mediates the immune response.

46. The method of claim 1 wherein the biological response is an immune response to an antigen or epitope that in recognized as self by the biological system.

47. The method of claim 46 wherein the multivalent ligand modifies the immune response to an antigen or epitope that is recognized as self and wherein the multivalent ligand comprises a signal recognition element that is an epitope or antigen that is recognized as self by the immune cell, cells or immune system.

48. The method of claim 1 wherein the biological system is an animal.

49. The method of claim 1 wherein the biological system is a mammal.

50. The method of claim 1 wherein the biological system is a human.

51. A method for treating a bacterial infection which comprises the step of administering a therapeutically effective amount of a multivalent ligand to an individual having a bacterial infection wherein the multivalent ligand comprises a plurality of signal recognition elements that are chemoattractant signals covalently bonded to a molecular scaffold.

52. A pharmaceutical composition for treating a bacterial infection which comprises an amount of a multivalent ligand effective for inhibiting the chemotaxis response in the bacterium, wherein the multivalent ligand comprises a plurality of signal recognition elements that are chemoattractant signals covalently bonded to a molecular scaffold, and a pharmaceutically acceptable carrier.

53. A method for modulating the chemotaxis response of a eukaryotic cell which comprises the step of contacting the eukaryotic cell with a multivalent ligand which comprises a plurality of signal recognition elements that are chemoattractants of the eukaryotic cell.

54. A method for treating an infection of a eukaryotic pathogen or parasite which comprises the step of administering a therapeutically effective amount of a multivalent ligand to an individual having an infection wherein the multivalent ligand comprises a plurality of signal recognition elements that are chemoattractants of the pathogen or parasite covalently bonded to a molecular scaffold.

55. A pharmaceutical composition for treating an infection of a eukaryotic pathogen or parasite which comprises an amount of a multivalent ligand effective for inhibiting the chemotaxis response in the pathogen or parasite, the multivalent ligand comprising a plurality of signal recognition elements that are chemoattractants covalently bonded to a molecular scaffold, and a pharmaceutically acceptable carrier.

56. The method of claim 1 wherein the response is cell migration, cell adhesion, or the formation of cell to cell junctions.

57. The method of claim 56 wherein the multivalent ligand inhibits cell migration, cell adhesion, or the formation of cell to cell junctions.

58. The method of claim 57 wherein the cell is a cancer cell in an animal.

59. The method of claim 1 wherein the multivalent ligand further comprises one or more recognition elements, one or more functional elements or both.

60. The method of claim 59 wherein one or more of the recognition elements binds to a protein.

61. The method of claim 59 wherein one or more of the functional elements is a label or a reporter group.

62. The method of claim 1 wherein one or more of the signal recognition elements is selected from the group consisting of an amino acid, a peptide, a protein, a derivatized peptide, a monosaccharide, a disaccharide, a polysaccharide, a nucleic acid, a cell nutrient, an epitope, an antigenic determinant, a hapten, or a cell surface receptor.

63. The method of claim 1 wherein one or more of the signal recognition elements is a saccharide or a derivatized saccharide.

64. The method of claim 1 wherein one or more of the signal recognition elements is a peptide or a derivatized peptide.

65. The method of claim 1 wherein one or more of the signal recognition elements is a protein.

66. The method of claim 1 wherein one or more of the signal recognition elements is an N-formyl peptide.

67. The method of claim 1 wherein one or more of the signal recognition elements is an epitope.

68. The method of claim 1 wherein the multivalent ligand comprises a defined number of signal recognition elements.

69. The method of claim 1 wherein the multivalent ligand comprises 2 to about 10 signal recognition elements.

70. The method of claim 1 wherein the multivalent ligand comprises about 10 to 25 signal recognition elements .

71. The method of claim 1 wherein the multivalent ligand comprises about 25 or more signal recognition elements .

72. The method of claim 1 wherein the multivalent ligand comprises about 50 or more signal recognition elements.

73. The method of claim 1 wherein the multivalent ligand comprises about 100 or more signal recognition elements.

74. The method of claim 1 wherein the signal recognition elements are covalently bonded to the molecular scaffold.

75. The method of claim 1 wherein the signal recognition elements are noncovalently bonded to the molecular scaffold.

76. The method of claim 75 wherein the multivalent ligand further comprises a plurality of recognition elements covalently bonded to the scaffold wherein the signal recognition elements are in turn noncovalently bonded to one or more recognition elements.

77. The method of claim 76 wherein the recognition elements are saccharides and the signal recognition elements are peptides which bind noncovalently to the saccharides.

78. The method of claim 77 wherein the signal recognition elements are lectins.

79. The method of claim 78 wherein the lectins are Concanavalin A.

80. The method of claim 1 wherein the molecular scaffold is selected from the group consisting of a polyacrylamide, a polyester, a polyether, a polymethacrylate, a polyol, and a polyamino acid.

81. The method of claim 1 wherein the molecular scaffold is a ROMP scaffold.

82. The method of claim 1 wherein the multivalent ligand has the structure: 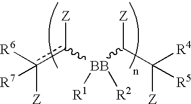
wherein:n is an integer that is 2 or more which represents the number of repeating units within the parentheses in the ligand;the dashed lines indicate optional double bonds; "BB"represents the backbone repeating unit, which may be cyclic or acyclic, and may be the same or different in a random or block arrangement, the wavy lines indicating that a BB unit may be in either a cis or trans configuration in the ligand backbone;each R¹ and R², independently of other R¹ and R² in the ligand, can be H or an organic group, a recognition element -L²-RE, a functional element -L³-FE or a signal recognition element -L¹-SRE or both of R¹ and R² can be the -L¹-SRE group;wherein L¹⁻³, independently, represent optional linker groups which may be the same or different in different repeating units;R⁴ and R⁵ are H, or an organic group;R⁶ and R⁷ are H, an organic group or an end-group; and Z , independently of other Z in the ligand, is H, OH, OR⁸, SH, a halide (F, Br, Cl, I), NH₂ or N(R⁸ )₂, where R⁸ is H or an organic group or Z is absent when the optional double bond is present.

83. The method of claim 82 wherein SRE is a peptide or a derivatized peptide.

84. The method of claim 83 wherein SRE is an N-formyl peptide.

85. The method of claim 82 wherein SRE is a chemoattractant.

86. The method of claim 82 wherein SRE is an epitope.

87. The method of claims 82 wherein at least one of SRE is an epitope and at least one other SRE binds to a cell surface receptor of an immune cell.

88. The method of claim 87 wherein the at least one other SRE binds to a cell surface receptor of a B-cell or a T-cell.

89. The method of claim 82 wherein at least one R¹ or R² is an -L³-FE group which is a detectable label or a reporter group.

90. The method of claim 82 wherein at least one R¹ or R² is an -L²-RE group.

91. The method of claim 82 wherein the multivalent ligand has the structure: 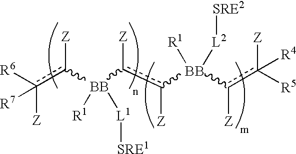
wherein: m + n is 2 or more;dashed lines indicate the presence of optional double bonds; "BB"represents the backbone repeating unit, which may be cyclic or acyclic, and may be the same or different in a random or block arrangement and wavy lines indicate that the BB unit may be in a cis or trans configuration in the backbone of the repeating unit;each R¹ , independent of other R¹ in the ligand, can be H or an organic group;L¹ and L², which may be the same or different, represent optional linker groups;SRE¹ and SRE² represent two different signal groups;R⁴ and R⁵ are H, an organic group or an end-group;R⁶ and R⁷ are H, an organic group or an end-group; and Z , independently of other Z in the polymer, is H, OH, OR⁸, SH, a halide (F, Br, Cl, I), NH₂ or N(R⁸ )₂ where R⁸ is H or an organic group or Z is absent when a double bond is present.

92. The method of claim 91 wherein one or both of SRE¹ and SRE² are peptides or derivatized peptides.

93. The method of claim 91 wherein one or both of SRE¹ or SRE² are saccharides or derivatized saccharides.

94. The method of claim 91 wherein one or both of SRE¹ or SRE² are epitopes.

95. The method of claim 91 wherein one of SRE¹ or SRE² is an epitope and the other of SRE¹ or SRE² binds to a cell surface receptor of an immune cell.

96. A multivalent ligand having the structure: 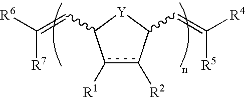
wherein:n is an integer that is 2 or more that represents the number of repeating units within the parentheses in the ligand; the dashed line indicates an optional double bondeach Y, independent of other Y in the ligand, is an -O-, a -S-, an -NR⁸, or a -CH₂- group, where R⁸ is H or an organic group;each R¹ and R², independent of other R¹ and R² in the ligand, can be H, an organic group, a signal recognition element -L¹-SRE, a recognition element -L²-RE or a functional element -L³-FE, wherein at least one of the R¹ and R² groups in the ligand is -L³-SRE;wherein L¹⁻³ represent optional linker groups;R⁴ and R⁵ are H, an organic group or an end-group; andR⁶ and R⁷ are H, an organic group or an end-group.

97. The multivalent ligand of claim 96 wherein one of the R¹ or R² groups in each repeating unit of the ligand is -L¹-SRE.

98. The multivalent ligand of claim 96 wherein at least one of the R¹ or R² groups in the ligand is -L²-RE .

99. The multivalent ligand of claim 96 wherein at least one of the R¹ or R² groups in the ligand is -L²-FE .

100. The multivalent ligand of claim 99 wherein the FE in the at least one -L²-FE group in the ligand is a detectable label or a reporter group.

101. The multivalent ligand of claim 99 wherein the FE in the at least one -L²-FE group in the ligand is an enzyme.

102. The multivalent ligand of claim 96 wherein at least one of SRE is a peptide or a derivatized peptide.

103. The multivalent ligand of claim 102 wherein at least one of SRE is an N-formyl peptide.

104. The multivalent ligand of claim 96 wherein at least one of SRE is an epitope.

105. The multivalent ligand of claim 104 wherein at least one of SRE binds to a cell surface receptor of an immune cell.

106. The multivalent ligand of claim 104 wherein at least one of SRE binds to a cell surface receptor of a B cell or a T cell.

107. The multivalent ligand of claim 96 having the structure: 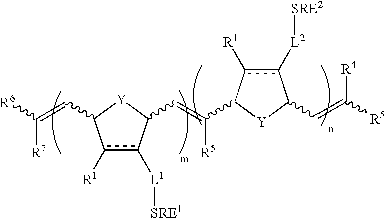
wherein: m + n is an integer of 2 or more and each integer represents the number of repeating units in the parentheses; each Y, independent of other Y in the ligand, is -O-, -S-, -NR⁸-, or -CH₂-;R¹ can be H, an organic group, a -L²-RE group or an -L³-FE group;L¹ and L², which may be the same or different, represent optional linker groups;SRE¹ and SRE² represent two different signal recognition elements;R⁴ and R⁵ are H, an organic group or an end-group; andR⁶ and R⁷ are H, an organic group or an end-group.

108. The multivalent ligand of claim 107 wherein one of SRE¹ or SRE² is a peptide or a derivatized peptide and the other of SRE¹ or SRE² is a saccharide.

109. The multivalent ligand of claim 108 wherein SRE¹ or SRE² are two different peptides or derivatized peptides.

110. The multivalent ligand of claim 107 wherein one of SRE¹ or SRE² is an epitope and the other of SRE¹ or SRE² binds to an immune cell.

111. The multivalent ligand of claim 110 wherein the immune cell is a B cell or a T cell.

112. The multivalent ligand of claim 111 wherein one of SRE¹ or SRE² is an epitope recognized as foreign by the B cell or T cell.

113. The multivalent ligand of claim 112 wherein one of SRE¹ or SRE² is an epitope recognized as self by the B cell or T cell.

114. The multivalent ligand of claim 113 wherein one of SRE¹ or SRE² binds to a CR2 receptor on a B cell.

115. The multivalent ligand of claim 114 wherein one of SRE¹ or SRE² binds to a CD22 receptor on a B cell.

116. A complex of a multivalent ligand of claim 96 with one or more proteins wherein in the multivalent ligand the at least one SRE groups binds to the protein.

117. The complex of claim 116 wherein the multivalent ligand comprises a plurality of SRE groups that bind to the protein.

118. The method of claim 116 wherein the protein is a lectin.

119. The complex of claim 118 wherein the SRE groups are monosaccharides.

120. The complex of claim 118 wherein the multivalent ligand is complexed to two or more lectin molecules.

121. The complex of claim 120 wherein the multivalent ligand is complexed to two or more concanavalin A molecules.

122. A method for enhancing aggregation of biological particles which comprises the steps of:
providing a multivalent ligand which comprises a plurality of recognition elements which each induce aggregation of one or more of the biological particles and contacting the biological particles with the complex.

123. The method of claim 122 wherein the recognition elements are antibodies or lectins.

124. The method of claim 122 wherein the biological particles are cells, viruses or virions.

125. The method of claim 122 wherein the multivalent ligand is a ROMP-derived ligand.

126. A method for inducing or enhancing induction of apoptosis in a cell which comprises the steps of:
forming a multivalent ligand which comprises a plurality of signal recognition elements which bind to the cell and induce apoptosis in the cell and contacting the cells with the multivalent ligand.

127. The method of claim 126 wherein one or more of the signal recognition elements is a lectin.

128. The method of claim 126 wherein the multivalent ligand is a ROMP-derived ligand.

129. The multivalent ligand of claim 96 which comprises a plurality of two or more different SRE.

130. The multivalent ligand of claim 129 wherein the SRE are bonded randomly to the molecular scaffold.

131. The multivalent ligand of claim 129 wherein the SRE are bonded in a selected pattern to the molecular scaffold.

132. The method of claim 122 wherein the multivalent ligand is bonded to a solid support.

133. A method for generating an assembly of biological macromolecules or particles which comprises the steps of:
(a)providing a multivalent ligand which comprises a molecular scaffold to which a plurality of recognition elements which, in turn, bind to one or more biological macromolecules or biological particles wherein the number, density and spacing of recognition elements bonded to the molecular scaffold are controlled.
(b)contacting the multivalent ligand with biological macromolecules or particles such that the recognition elements of the ligand bind to two or more biological macromolecules or biological particles.

134. The method of claim 133 wherein the biological macromolecules are peptides.

135. The method of claim 133 wherein the biological particles are cells, viruses or virions.

136. The method of claim 133 wherein the multivalent ligand further comprises one or more FE bonded to the molecular scaffold.

137. The method of claim 133 wherein the FE is a detectable label.

138. The method of claim 133 wherein the FE is a group that can be attached to a solid support.

139. The method of claim 133 wherein the molecular scaffold is a polymer.

140. The method of claim 1 wherein the multivalent ligand is bonded to a solid support.

141. The method of claim 82 wherein the multivalent ligand is bonded to a solid support.
